SEQUENCE LISTING (8 sequences)

SEQ ID NO: 1 - DNA, 16360 bp, Artificial Sequence
  Description of Artificial Sequence/note = synthetic construct
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatccattcgggagggcatagcatcaatagatgtgctgcccctcagtccgttgatacctacttggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggtacccctgtaaacaatgctggactatttaaaaatccttgaggaagacgtgtggtggaatgccactagaaactagggaaaactaggaggagagtattacagggaaggaagtgaagaacctcgtgacccaaatactcctgctcctcatagacgtacctgggatgagagacacaaggttcttaaattgtcctcattcgctactccctctgacatccaacgctgggctactaaagcattgccttatggctggaaagtggtcaccgaaagcggaaatgattatactagccgcagaaagatcagaacattgacagagatgactcaggatgaaattagaaaaaggtgggaaagtggatattgtgaccccttcattgactcaggaagtgactcagatggacccttctaaaagccacagacagtaaaaatgtgttagcactttatacaatattatatctgcttaagctatagaagctttcacatactcagtagctgtttcacaatcaacaaaacaatgatgatgtaatcataaggaagtagtttaaaataggttaataagtttattagttatatagaaaataatataggataaagtataaggattaaggtatgaggtgtgtggctcaacacgtagggtgacaagaaaatctactgtaataggacacaacacctctaaagttgcccgtgggaaggtgaagtgagatcgaatctttccttaacgcagacagctttttatccactagggataatgttttaaggaatactatagtaatagattgatagttttaacaatgatggaaatagtatataaggatagtttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggcttcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgtctaatattacaagatgatgataatgaacctttacagagacctagatatgaggtaatacaacgagctgtaaaccctcatacaatgtttatgatatcaggaccattagctgaacttcaattagcctttcaggatttagatttacctgaaggtccattgaggtttggtccattggcaaatggacattatgttcaaggagatccttatagtagttcttacagaccagtaacaatggccgaaacagcccaaatgactagagatgaactggaagatgttcttaatactcaaagtgaaatagaaattcaaatgataaatttattggagttgtatgaagttgaaactagagctcttagaagacaattagctgagagatctagtacagggcaaggaggaatatccccaggagctcctcgttctcgaccaccagtaagcagcttctcagggttaccaagtttgccctctatacctgggattcatcccagggcaccttcacctccaagggcaacttctactcccggaaatattccttggagtttaggagatgatagcccaccttcatctagttttcctggaccctctcaacctcgtgtttctttccatccgggaaatccttttgttgaagaagaaggtcatagacctagatcccagtctagagaaaggagaagagaaattcttcctgctcctgtaccgtcagcacctcctatgattcagtatataccagtaccacctccaccaccgattggcacggttatacctattcagcatatcagatctgtaactggagagcctcctagaaacccaagagaaataccaatttggctaggacgaaatgctcctgctatagatggagtgttccctgttacaacaccggatctaagatgcagaataattaatgctatactaggaggaaatattgggctatcattaacccctggagactgtttaacatgggactcagcagtagccaccttatttattagaacccatggaacttttccaatgcatcagcttggaaatgtaataaaaggcatagttgatcaagaaggagtggcaacagcatatactttgggaatgatgctttctggacaaaattatcaattagtttctggaataattagaggatatttgcctggacaagctgtagtaactgcattacaacagcgtttagaccaagaaatagataatcaaacaagagcagagacttttattcaacatctaaatgctgtatatgaaattttaggccttaatgccagaggacaaagtatacgtgcttcagtgactcctcaaccccgaccatccagaggtagaggtcgaggtcaaaatacttctagaccctctcaaggaccagctaatagcgggcggggacgacagcgccctgcttctggtcaaagcaacagaggatctagtactcagaatcaaaatcaagataatttaaatcaaggaggatataatcttcgaccccgtacttaccaacctcaaaggtacggaggaggacgtggacgaagatggaacgataatactaacaatcaagagtccagaccatcagatcaaggttctcaaactcctaggccaaatcaagcaggctctggggtgcgtggcaatcagtcacaaactcccagaccagctgctggtcgcggaggaagaggtaaccacaaccgaaaccaacgatcatccggtgctggtgactcacgcgctgtcaataccgtgacacagagtgccacgtcctccacagatgaatcctcttcagctgttacagccgcttccggcggagatcaaagggactaaattgttagcccactgggattcaggggcaacaataacttgtattcctgaaagttttttagaagatgaacaacctattaaaaagactttaataaaaacaattcatggagaaaaacaacaaaatgtttattatgtaacctttaaagttaaaggaagaaaagtggaagcagaagtgatagcttctccttatgagtatattttgctgtcgccaacagatgttccttggttaacacagcaaccacttcagttaacaattttagttcctcttcaagaatatcaagagaaaatcttaagtaagactgctcttccagaagatcaaaaacaacaattaaaaaccttgtttgtcaagtatgacaatctatggcaacattgggaaaatcaagtcgggcatagaaaaattaggccacataatatagcaactggtgattatcctcctcgccctcaaaaacaatatcctattaatcctaaggcaaagcctagtatacaaattgtaatagatgacttattgaaacaaggggtgttaacgcctcaaaatagtacaatgaatacaccagtgtatcctgttcctaaaccagatggaaggtggagaatggtattagattatagagaagtaaataaaactattccattaacagctgcccaaaaccaacactctgctggtattttagctactattgttagacaaaaatataaaactaccttagatttagctaatggattttgggctcatcctattacaccagaatcttattggttaacagcatttacctggcaaggtaaacagtattgttggacacgtcttcctcaaggatttttaaatagtccagcattgtttacagctgatgtagtagatttactaaaagaaatccctaatgtacaagtgtatgttgatgatatatatttaagccatgatgatcctaaagagcatgttcaacaattagaaaaagtgtttcaaattttactacaggcaggatatgtagtatctttgaaaaaatcagaaattggtcaaaaaactgtagaatttttaggatttaatattactaaagaaggtcgtggcctaacagacacttttaaaacaaaactgttaaatattactcctccaaaagacttaaagcaattacaaagcatattaggattgttaaattttgctagaaattttatacctaattttgctgaactggtacaaccattatacaatttaatagcctcagcaaaaggcaaatatattgagtggtctgaagaaaatactaaacaattaaatatggtaatagaagcattaaacactgcctctaatttagaagaaaggttaccagaacagagactggtaattaaagtcaatacttctccatcagcaggatatgtaagatattataatgagactggtaaaaagcctattatgtacctaaattatgtgttttccaaagcagaattaaaattttctatgttagaaaaactattaactacaatgcacaaagccttaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggaacccatagtggtgaaggagagggtcctaggtctttctcaaattctgatgattaattcagaaaacattgctaataatgctaatttgacacaagaagtaaagaagttgttaactgaaatggttaatgaagaaatgcaaagtttgtcagatgtaatgattgactttgaaattcctttaggagaccctcgtgatcaagaacaatatatacatagaaaatgctatcaagaatttgcaaattgttatttagtaaaatataaagaacccaaaccgtggcctaaggagggccttatagctgatcaatgcccattaccaggttaccatgctggattaacctataatagacagtctatttgggattactatattaaagtggagagtattagacctgcaaattggacaacaaagagtaaatatggacaagctagactaggaagtttttatattcctagcagcctgagacaaatcaatgttagtcatgtactattctgtagtgatcaattatattctaaatggtataatatagaaaataccatagaacaaaacgagcggtttctgcttaataaactaaataaccttacatctggaacctcagtattgaagaaaagagctcttccgaaggattggagttctcaaggtaaaaatgctctgtttagagaaatcaatgtgttagatatctgcagtaaacctgaatctgtaatactattgaatacttcatactattccttctctttatgggaaggagattgtaattttactaaagatatgatttctcagttggttccagaatgtgatggattttataacaattctaagtggatgcatatgcatccatatgcttgtagattctggagaagtaagaagaatgaaaaagaagaaactaaatgtagagatggggaaactaagagatgtctgtattatcctttatgggacagtcccgaatctacatatgattttggttatttagcataccaaaagaattttccttcccctatctgtatagaacaacagaaaattagagatcaagattatgaagtctattctttgtatcaagaacgcaaaatagcttctaaagcatatggaattgatacagttttattctctctaaagaattttcttaattatacaggaactcctgtaaatgaaatgcctaatgcaagagcttttgtaggcctaatagatcccaagtttcctccttcctatcccaatgttactagggaacattatacttcctgtaataataggaaaagaagaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgaaaaagaagaatcagtagcctccacctctggaattcaagacctgcagactctgagtgagcttgttggtcctgaaaatgccggagagggagagctgactattgctgaggaacctgaagaaaatcctcgacgccccagacgatatactaaaagagaagtcaaatgtgtgtcttatcatgcatataaagaaattgaggacaaacatcctcaacatattaaactgcaggattggatccccacaccagaggaaatgagtaagtcactctgtaaaagacttattttatgtggattgtatagtgcagaaaaggcctcagagattttaaggatgccttttacagtttcttgggaacaatcagatactgaccctgactgttttattgtaagctatacatgtatattttgtgatgctgtaatacatgatcccatgcccataagatgggatcctgaagttggaatttgggtaaaatataaacccctcagaggaattgttggatctgctgtgtttattatgcataaacatcaaagaaactgttctcttgttaaaccttctaccagtcgctcagaaggtccaaaaccaagacctaggcacgatcctgtccttcgatgtgacatgtttgaaaagcatcacaagcctcggcagaaacgacccaggagacgatccatcgataatgagtcatgtgcttccagtagtgacaccatggccaatgagccaggatcactatgcaccaaccctctttggaatcctggaccgctactatcagggctacttgaagagtccagcaacctaccaaacttggaagttcacatgtcaggtggacccttctgggaagaggtttatggggactcaattttgggtcccccctctgggtcaggtgaacattcagttttataagaattatcagattctaacttgctgtcaggctgtagatcttcttaagcttgcgggagacgtcgagtccaaccctgggcccgatatccccatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttgatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttgtaaagcggccgcgactctaggggattcgcgataagtaagtaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaaagcattgccttatggctggaaagtggtcaccgaaagcggaaatgattatactagccgcagaaagatcagaacattgacagagatgactcaggatgaaattagaaaaaggtgggaaagtggatattgtgaccccttcattgactcaggaagtgactcagatggacccttctaaaagccacagacagtaaaaatgtgttagcactttatacaatattatatctgcttaagctatagaagctttcacatactcagtagctgtttcacaatcaacaaaacaatgatgatgtaatcataaggaagtagtttaaataggttaataagtttattagttatatagaaaataatataggataaagtataaggattaaggtatgaggtgtgtggctcaacacgtagggtgacaagaaaatctactgtaataggacacaacacctctaaagttgcccgtgggaaggtgaagtgagatcgaatctttccttaacgcagacagctttttatccactagggataatgttttaaggaatactatagtaatagattgatagttttaacaatgatggaaatagtatataaggatagtttctagattgtacgggaggctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctag

SEQ ID NO: 2 - DNA, 1503 bp, Artificial Sequence
  Description of Artificial Sequence/note = synthetic construct
atgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 3 - protein, 500 aa, Artificial Sequence
  Description of Artificial Sequence/note = synthetic construct
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 4 - protein, 512 aa, Artificial Sequence
  Description of Artificial Sequence/note = synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHIARNCRAPRKKGCWKCGKEGHQMKDCTERQANFLGKIWPSYKGRPGNFLQSRPEPTAPPFLQSRPEPTAPPEESFRSGVETTTPSQKQEPIDKELYPLTSLRSLFGNDPSSQ

SEQ ID NO: 5 - protein, 512 aa, Artificial Sequence
  Description of Artificial Sequence/note = synthetic construct
MGARASVLSGGELDRWEKVRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHIARNCRAPRKKGCWKCGKEGHQMKDCTERQANFLGKIWPSYKGRPGNFLQSRPEPTAPPFLQSRPEPTAPPEESFRSGVETTTPSQKQEPIDKELYPLTSLRSLFGNDPSSQ

SEQ ID NO: 6 - DNA, 34 bp, Artificial Sequence
  Description of Artificial Sequence/note = synthetic construct
tccgggcccggaatgcctatagtccagaacatcc

SEQ ID NO: 7 - DNA, 29 bp, Artificial Sequence
  Description of Artificial Sequence/note = synthetic construct
gcggccgcgttttgagaacgaaataccgg

SEQ ID NO: 8 - DNA, 17207 bp, Artificial Sequence
  Description of Artificial Sequence/note = synthetic construct
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatccattcgggagggcatagcatcaatagatgtgctgcccctcagtccgttgatacctacttggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggtacccctgtaaacaatgctggactatttaaaaatccttgaggaagacgtgtggtggaatgccactagaaactagggaaaactaggaggagagtattacagggaaggaagtgaagaacctcgtgacccaaatactcctgctcctcatagacgtacctgggatgagagacacaaggttcttaaattgtcctcattcgctactccctctgacatccaacgctgggctactaaagcattgccttatggctggaaagtggtcaccgaaagcggaaatgattatactagccgcagaaagatcagaacattgacagagatgactcaggatgaaattagaaaaaggtgggaaagtggatattgtgaccccttcattgactcaggaagtgactcagatggacccttctaaaagccacagacagtaaaaatgtgttagcactttatacaatattatatctgcttaagctatagaagctttcacatactcagtagctgtttcacaatcaacaaaacaatgatgatgtaatcataaggaagtagtttaaaataggttaataagtttattagttatatagaaaataatataggataaagtataaggattaaggtatgaggtgtgtggctcaacacgtagggtgacaagaaaatctactgtaataggacacaacacctctaaagttgcccgtgggaaggtgaagtgagatcgaatctttccttaacgcagacagctttttatccactagggataatgttttaaggaatactatagtaatagattgatagttttaacaatgatggaaatagtatataaggatagtttctagattgtacgggagctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaacctactcatatattattgtctcttttatactttattaagtaaaaggatttgtatattagccttgctaagggagacatctagtgatataagtgtgaactacacttatcttaaatgatgtaactccttaggataatcaatatacaaaattccatgacaattggcgcccaacgtggggctcgaatataagtcgggtttatttgtaaattatccctagggacctccgagcatagcgggaggcatataaaagccaatagacaatggcttcaggaagtaatgttgaagaatatgaacttgatgttgaagctctggttgtaattttaagagatagaaatataccaagaaatcctttacatggagaagttataggtcttcgccttactgaaggatggtggggacaaattgagagatttcagatggtacgtctaatattacaagatgatgataatgaacctttacagagacctagatatgaggtaatacaacgagctgtaaaccctcatacaatgtttatgatatcaggaccattagctgaacttcaattagcctttcaggatttagatttacctgaaggtccattgaggtttggtccattggcaaatggacattatgttcaaggagatccttatagtagttcttacagaccagtaacaatggccgaaacagcccaaatgactagagatgaactggaagatgttcttaatactcaaagtgaaatagaaattcaaatgataaatttattggagttgtatgaagttgaaactagagctcttagaagacaattagctgagagatctagtacagggcaaggaggaatatccccaggagctcctcgttctcgaccaccagtaagcagcttctcagggttaccaagtttgccctctatacctgggattcatcccagggcaccttcacctccaagggcaacttctactcccggaaatattccttggagtttaggagatgatagcccaccttcatctagttttcctggaccctctcaacctcgtgtttctttccatccgggaaatccttttgttgaagaagaaggtcatagacctagatcccagtctagagaaaggagaagagaaattcttcctgctcctgtaccgtcagcacctcctatgattcagtatataccagtaccacctccaccaccgattggcacggttatacctattcagcatatcagatctgtaactggagagcctcctagaaacccaagagaaataccaatttggctaggacgaaatgctcctgctatagatggagtgttccctgttacaacaccggatctaagatgcagaataattaatgctatactaggaggaaatattgggctatcattaacccctggagactgtttaacatgggactcagcagtagccaccttatttattagaacccatggaacttttccaatgcatcagcttggaaatgtaataaaaggcatagttgatcaagaaggagtggcaacagcatatactttgggaatgatgctttctggacaaaattatcaattagtttctggaataattagaggatatttgcctggacaagctgtagtaactgcattacaacagcgtttagaccaagaaatagataatcaaacaagagcagagacttttattcaacatctaaatgctgtatatgaaattttaggccttaatgccagaggacaaagtatacgtgcttcagtgactcctcaaccccgaccatccagaggtagaggtcgaggtcaaaatacttctagaccctctcaaggaccagctaatagcgggcggggacgacagcgccctgcttctggtcaaagcaacagaggatctagtactcagaatcaaaatcaagataatttaaatcaaggaggatataatcttcgaccccgtacttaccaacctcaaaggtacggaggaggacgtggacgaagatggaacgataatactaacaatcaagagtccagaccatcagatcaaggttctcaaactcctaggccaaatcaagcaggctctggggtgcgtggcaatcagtcacaaactcccagaccagctgctggtcgcggaggaagaggtaaccacaaccgaaaccaacgatcatccggtgctggtgactcacgcgctgtcaataccgtgacacagagtgccacgtcctccacagatgaatcctcttcagctgttacagccgcttccggcggagatcaaagggactaaattgttagcccactgggattcaggggcaacaataacttgtattcctgaaagttttttagaagatgaacaacctattaaaaagactttaataaaaacaattcatggagaaaaacaacaaaatgtttattatgtaacctttaaagttaaaggaagaaaagtggaagcagaagtgatagcttctccttatgagtatattttgctgtcgccaacagatgttccttggttaacacagcaaccacttcagttaacaattttagttcctcttcaagaatatcaagagaaaatcttaagtaagactgctcttccagaagatcaaaaacaacaattaaaaaccttgtttgtcaagtatgacaatctatggcaacattgggaaaatcaagtcgggcatagaaaaattaggccacataatatagcaactggtgattatcctcctcgccctcaaaaacaatatcctattaatcctaaggcaaagcctagtatacaaattgtaatagatgacttattgaaacaaggggtgttaacgcctcaaaatagtacaatgaatacaccagtgtatcctgttcctaaaccagatggaaggtggagaatggtattagattatagagaagtaaataaaactattccattaacagctgcccaaaaccaacactctgctggtattttagctactattgttagacaaaaatataaaactaccttagatttagctaatggattttgggctcatcctattacaccagaatcttattggttaacagcatttacctggcaaggtaaacagtattgttggacacgtcttcctcaaggatttttaaatagtccagcattgtttacagctgatgtagtagatttactaaaagaaatccctaatgtacaagtgtatgttgatgatatatatttaagccatgatgatcctaaagagcatgttcaacaattagaaaaagtgtttcaaattttactacaggcaggatatgtagtatctttgaaaaaatcagaaattggtcaaaaaactgtagaatttttaggatttaatattactaaagaaggtcgtggcctaacagacacttttaaaacaaaactgttaaatattactcctccaaaagacttaaagcaattacaaagcatattaggattgttaaattttgctagaaattttatacctaattttgctgaactggtacaaccattatacaatttaatagcctcagcaaaaggcaaatatattgagtggtctgaagaaaatactaaacaattaaatatggtaatagaagcattaaacactgcctctaatttagaagaaaggttaccagaacagagactggtaattaaagtcaatacttctccatcagcaggatatgtaagatattataatgagactggtaaaaagcctattatgtacctaaattatgtgttttccaaagcagaattaaaattttctatgttagaaaaactattaactacaatgcacaaagccttaattaaggctatggatttggccatgggacaagaaatattagtttatagtcccattgtatctatgactaaaatacaaaaaactccactaccagaaagaaaagctttacccattagatggataacatggatgacttatttagaagatccaagaatccaatttcattatgataaaaccttaccagaacttaagcatattccagatgtatatacatctagtcagtctcctgttaaacatccttctcaatatgaaggagtgttttatactgatggctcggccatcaaaagtcctgatcctacaaaaagcaataatgctggcatgggaatagtacatgccacatacaaacctgaatatcaagttttgaatcaatggtcaataccactaggtaatcatactgctcagatggctgaaatagctgcagttgaatttgcctgtaaaaaagctttaaaaatacctggtcctgtattagttataactgatagtttctatgtagcagaaagtgctaataaagaattaccatactggaaatctaatgggtttgttaataataagaaaaagcctcttaaacatatctccaaatggaaatctattgctgagtgtttatctatgaaaccagacattactattcaacatgaaaaaggcatcagcctacaaataccagtattcatactgaaaggcaatgccctagcagataagcttgccacccaaggaagttatgtggttaattgtaataccaaaaaaccaaacctggatgcagagttggatcaattattacagggtcattatataaaaggatatcccaaacaatatacatattttttagaagatggcaaagtaaaagtttccagacctgaaggggttaaaattattccccctcagtcagacagacaaaaaattgtgcttcaagcccacaatttggctcacaccggacgtgaagccactcttttaaaaattgccaacctttattggtggccaaatatgagaaaggatgtggttaaacaactaggacgctgtcaacagtgtttaatcacaaatgcttccaacaaagcctctggtcctattctaagaccagataggcctcaaaaaccttttgataaattctttattgactatattggacctttgccaccttcacagggatacctatatgtattagtagttgttgatggaatgacaggattcacttggttataccccactaaggctccttctactagcgcaactgttaaatctctcaatgtactcactagtattgcaattccaaaggtgattcactctgatcaaggtgcagcattcacttcttcaacctttgctgaatgggcaaaggaaagaggtatacatttggaattcagtactccttatcacccccaaagtggtagtaaggtggaaaggaaaaatagtgatataaaacgacttttaactaaactgctagtaggaagacccacaaagtggtatgacctattgcctgttgtacaacttgctttaaacaacacctatagccctgtattaaaatatactccacatcaactcttatttggtatagattcaaatactccatttgcaaatcaagatacacttgacttgaccagagaagaagaactttctcttttacaggaaattcgtacttctttataccatccatccacccctccagcctcctctcgttcctggtctcctgttgttggccaattggtccaggagagggtggctaggcctgcttctttgagacctcgttggcataaaccgtctactgtacttaaggtgttgaatccaaggactgttgttattttggaccatcttggcaacaacagaactgtaagtatagataatttaaaacctacttctcatcagaatggcaccaccaatgacactgcaacaatggatcatttggaaaaaaatgaataaagcgcatgaggcacttcaaaatacaacaactgtgactgaacagcagaaggaacaaattatactggacattcaaaatgaagaagtacaaccaactaggagagataaatttagatatctgctttatacttgttgtgctactagctcaagagtattggcctggatgtttttagtttgtatattgttaatcattgttttggtttcatgctttgtgactatatccagaatacaatggaataaggatattcaggtattaggacctgtaatagactggaatgttactcaaagagctgtttatcaacccttacagactagaaggattgcacgttcccttagaatgcagcatcctgttccaaaatatgtggaggtaaatatgactagtattccacaaggtgtatactatgaaccccatccggaacccatagtggtgaaggagagggtcctaggtctttctcaaattctgatgattaattcagaaaacattgctaataatgctaatttgacacaagaagtaaagaagttgttaactgaaatggttaatgaagaaatgcaaagtttgtcagatgtaatgattgactttgaaattcctttaggagaccctcgtgatcaagaacaatatatacatagaaaatgctatcaagaatttgcaaattgttatttagtaaaatataaagaacccaaaccgtggcctaaggagggccttatagctgatcaatgcccattaccaggttaccatgctggattaacctataatagacagtctatttgggattactatattaaagtggagagtattagacctgcaaattggacaacaaagagtaaatatggacaagctagactaggaagtttttatattcctagcagcctgagacaaatcaatgttagtcatgtactattctgtagtgatcaattatattctaaatggtataatatagaaaataccatagaacaaaacgagcggtttctgcttaataaactaaataaccttacatctggaacctcagtattgaagaaaagagctcttccgaaggattggagttctcaaggtaaaaatgctctgtttagagaaatcaatgtgttagatatctgcagtaaacctgaatctgtaatactattgaatacttcatactattccttctctttatgggaaggagattgtaattttactaaagatatgatttctcagttggttccagaatgtgatggattttataacaattctaagtggatgcatatgcatccatatgcttgtagattctggagaagtaagaagaatgaaaaagaagaaactaaatgtagagatggggaaactaagagatgtctgtattatcctttatgggacagtcccgaatctacatatgattttggttatttagcataccaaaagaattttccttcccctatctgtatagaacaacagaaaattagagatcaagattatgaagtctattctttgtatcaagaacgcaaaatagcttctaaagcatatggaattgatacagttttattctctctaaagaattttcttaattatacaggaactcctgtaaatgaaatgcctaatgcaagagcttttgtaggcctaatagatcccaagtttcctccttcctatcccaatgttactagggaacattatacttcctgtaataataggaaaagaagaagtgttgataataactatgctaagttaaggtctatggggtatgcacttacaggagcagtgcaaaccttatctcaaatatcagatattaatgatgaaaacttacagcaaggaatatatttattaagggatcatgtaataaccttaatggaagctacattgcatgatatatctgttatggaaggaatgtttgctgtacaacatttgcatacacatttgaatcatttgaagacaatgcttctagaaagaagaatagactggacctatatgtctagtacttggctacaacaacaattacagaaatctgatgatgagatgaaagtaataaagagaattgctagaagtttggtatattatgttaaacaaacccatagttctcccacagctacagcctgggagattggattatattatgaattggttatacctaaacatatttacttgaataattggaatgttgtcaatataggtcacttagttaaatcagctggacaattgactcatgtaactatagctcatccttatgaaataatcaataaggaatgtgtagagactatatatctgcatcttgaggactgcacaagacaagattatgtcatatgtgatgtggtaaagatagtgcagccttgtggcaatagctcagacacgagtgattgtcctgtctgggctgaagctgtaaaagaaccatttgtgcaagtcaatcctctgaaaaacggaagttatctggttttggcaagttccacagactgtcagatcccaccatatgttcctagcatcgtgactgttaatgaaacaacgtcatgctttggactggactttaaaaggccactggttgcggaagaaagattgagctttgagccacgactgccaaatctacaactaagattaccacatttggttggaattattgcaaaaatcaaagggataaaaatagaagtcacatcctctggagaaagtataaaagagcagattgaaagagcaaaagctgagctccttcgactggacattcacgagggagatactcctgcctggatacaacagctagctgcagcaacaaaggacgtctggccagcagcagcttctgctctacaaggaattggtaactttttatctgggactgcccaaggaatatttggaactgcctttagtctcttgggatacttaaagcctatcctaataggagtaggggtcattctcttggttattcttatatttaagattgtatcatggattcctacgaaaaagaagaatcagtagcctccacctctggaattcaagacctgcagactctgagtgagcttgttggtcctgaaaatgccggagagggagagctgactattgctgaggaacctgaagaaaatcctcgacgccccagacgatatactaaaagagaagtcaaatgtgtgtcttatcatgcatataaagaaattgaggacaaacatcctcaacatattaaactgcaggattggatccccacaccagaggaaatgagtaagtcactctgtaaaagacttattttatgtggattgtatagtgcagaaaaggcctcagagattttaaggatgccttttacagtttcttgggaacaatcagatactgaccctgactgttttattgtaagctatacatgtatattttgtgatgctgtaatacatgatcccatgcccataagatgggatcctgaagttggaatttgggtaaaatataaacccctcagaggaattgttggatctgctgtgtttattatgcataaacatcaaagaaactgttctcttgttaaaccttctaccagtcgctcagaaggtccaaaaccaagacctaggcacgatcctgtccttcgatgtgacatgtttgaaaagcatcacaagcctcggcagaaacgacccaggagacgatccatcgataatgagtcatgtgcttccagtagtgacaccatggccaatgagccaggatcactatgcaccaaccctctttggaatcctggaccgctactatcagggctacttgaagagtccagcaacctaccaaacttggaagttcacatgtccgatatccccatgggtgcgagagcgtcggtattaagcgggggagaattacaggtggacccttctgggaagaggtttatggggactcaattttgggtcccccctctgggtcaggtgaacattcagttttataagaattatcagattctaacttgctgtcaggctgtagatcttcttaagcttgcgggagacgtcgagtccaaccctatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataacaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttgatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttgtaaagcggccgcgactctaggggattcgcgataagtaagtaagcttatggacctcagagaggaagtaacgaggagagggtgtggtggaatgtcactagaaaccagggaaaacaaggaggagagtattacagggaaggaggtgaagaacctcattacccaaatactcctgctcctcatagacgtacctgggatgagagacacaaggttcttaaattgtcctcattcgctactccctctgacatccaacgctgggctactaaagcattgccttatggctggaaagtggtcaccgaaagcggaaatgattatactagccgcagaaagatcagaacattgacagagatgactcaggatgaaattagaaaaaggtgggaaagtggatattgtgaccccttcattgactcaggaagtgactcagatggacccttctaaaagccacagacagtaaaaatgtgttagcactttatacaatattatatctgcttaagctatagaagctttcacatactcagtagctgtttcacaatcaacaaaacaatgatgatgtaatcataaggaagtagtttaaataggttaataagtttattagttatatagaaaataatataggataaagtataaggattaaggtatgaggtgtgtggctcaacacgtagggtgacaagaaaatctactgtaataggacacaacacctctaaagttgcccgtgggaaggtgaagtgagatcgaatctttccttaacgcagacagctttttatccactagggataatgttttaaggaatactatagtaatagattgatagttttaacaatgatggaaatagtatataaggatagtttctagattgtacgggaggctcttcactactcgctgcgtcgagagtgtacgagactctccaggtttggtaagaaatattttatattgttataatgttactatgatccattaacactctgcttatagattgtaagggtgattgcaatgctttctgcataaaactttggttttcttgttaatcaataaaccgacttgattcgagaaccaactcctatattattgtctcttttatactttattaagtaaaaggatttgtatattagccttgctaagggagacatctagtgatataagtgtgaactacacttatcttaaatgatgtaactccttaggataatcaatatacaaaattccatgacaagatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctag

(19) United States
(12) Patent Application Publication
(10) Pub. No.: US 2005/0244429 A1
(43) Pub. Date: Nov. 3, 2005

(54) Live replicating spumavirus vector

(76) Inventors: Thomas Folks, Lithonia, GA (US); Irvin S. Y. Chen, Palos Verde Estates, CA (US)

Correspondence Address: CENTERS FOR DISEASE CONTROL;c/o NEEDLE & ROSENBERG P.C., 999 PEACHTREE STREET, SUITE 1000, ATLANTA, GA, 30309, US

(21) Appl. No.: 10/519,531

(22) PCT Filed: Jun. 27, 2003
(86) PCT No.: PCT/US03/20325

§ 371 Date: May 31, 2005

Related U.S. Application Data
(60) Provisional application No. 60/392,630, filed on Jun. 27, 2002.

(51) Int. Cl. A61K 39/12; C12N 7/00; C12N 15/867

(52) U.S. Cl. 424/199.1; 435/235.1; 435/456

(57) ABSTRACT

The present invention provides a vector or vector containing composition comprising a spumavirus backbone and a antigen-encoding nucleic acid. The present invention also provides methods of treating or preventing a condition resulting from a vital, bacterial, or parasitic infection in a subject comprising administering to the subject an effective amount of the vector or vector containing composition comprising a spumavirus backbone and an antigen-encoding nucleic acid. Also provided in the present invention are methods of treating a condition resulting from a cancer in a subject comprising administering to the subject an effective amount of the vector or vector containing composition comprising a spumavirus backbone and an antigen-encoding nucleic acid.

### BACKGROUND OF THE INVENTION

1. Spumavirus, also known as foamy virus for the characteristics of vacuolization the virus induces in cell culture, belongs to a distinct group of retroviruses. The simian foamy viruses (SFVs) include isolates from Old World and New World monkeys and are classified into 10 different serotypes based on serological cross-reactivities. Virus appears to persist in the host for a long period of time in a latent form and can exist in the presence of neutralizing antibody.

2. Currently the most studied retrovirus, Human Immunodeficiency Virus, is believed to be derived from nonhuman primate transmission into humans at some past time. Concerns about the risk of transmission of retroviruses from non-human primates to humans working in research laboratories were heightened in the early 1990's when two persons developed antibodies to SIV (Simian Immunodeficiency Virus) following work-related exposures, one of whom had clear evidence of persistent viral infection. (See CDC anonymous survey for simian immunodeficiency virus (SIV) seropositivity in SIV laboratory researchers—United States, 1992. MMWR Morb. Mort. Wkly. Rep. 1992; 41:814-5; Khabbaz R. F., et al. Brief report: infection of a laboratory worker with simian immunodeficiency virus. New Eng. J. Med. 1994; 330:172-7; Khabbaz R. F., et al. Simian immunodeficiency virus needle stick accident in a laboratory worker. Lancet 1992; 340:271-3; and CDC. Guideline to prevent simian immunodeficiency virus infection in laboratory workers and animal handlers. MMWR 1988; 37:693-704.) In addition to SIV, nonhuman primate species used in biomedical research are commonly infected with SFV (simian foamy virus), STLV (simian t-cell lymphotrophic virus), and/or type D retroviruses. All of these retroviruses cause lifelong infections in nonhuman primates, and some are known to be transmissible through sexual contact, blood, or breast-feeding. Natural SFV infections in non-human primates have not been definitively associated with disease. In non-human primates, infection with the other retroviruses may result in a clinical spectrum ranging from asymptomatic infection to life threatening immunodeficiency syndromes or lymphoproliferative disorders. The transmission routes of SFVs among non-human primates remain undefined, but the prevalence of seroreactivity is high among captive adult non-human primates.

3. Recent publications indicate that earlier serological tests showing human spumavirus antibodies in the human populating were incorrect. Immunological investigation of a previously reported human spumavirus revealed that it shared common antigens in complement fixation, immunofluorescence and neutralization assays with the chimpanzee foamy virus, SFV-6. Furthermore, failure to detect serological evidence of HFV infection in people from a wide geographical area suggested that this virus isolate was a variant of SFV-6 particularly since sera from chimpanzees naturally infected with SFV-6 neutralized both viruses. In a survey for prevalence of human foamy virus in more than 5000 human sera, collected from geographically diverse populations, none of the serum samples were confirmed as positive. Taken together with sequence analysis endorsing the phylogenetic closeness of the purported human spumavirus to SFV-6/7, these data strongly suggest that human foamy virus is not naturally found in the human populations. (See Ali, M. et al., “No Evidence of Antibody to Human Foamy Virus in Widespread Human Populations,” AIDS Research and Human Retroviruses, Vol. 12, NO. 15, 1996).

4. Gene therapies have long looked for a good vector that can transport the foreign gene of choice into human cells. Thus, compositions and methods for gene therapy are needed that use a vector capable of carrying a significant amount of foreign DNA that will enter the host organism and not cause disease.

5. Compositions and methods for vaccination using recombinant live retroviruses are also needed. A live virus, that causes no illness in humans, and that has genes of antigens of choice incorporated into its genome, would provide for an excellent vaccination tool as a vector. The retrovirus would reproduce in the human host and expose the immune system to antigens so that an immune response can be initiated.

6. Targeted attack on reproducing cells is a goal of cancer treatment. What is needed are compositions and methods for cancer treatment that are specific for dividing cells that do not cause systemic damage to the cancer patient. A viral vector that could infect and kill dividing cells, without killing other cells of the host would provide a solution for cancer treatment.

7. The lack of any known disease associated with the virus from which the vector of the present invention was derived makes the present invention ideal for gene therapy regimens.

### SUMMARY OF THE INVENTION

8. In accordance with the purposes of this invention, as embodied and broadly described herein, this invention, in one aspect, relates to live replicating retroviral vectors and methods of their use.

9. Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

10. The present invention provides compositions comprising live replicating retroviral vectors, wherein the vector is derived from a spumavirus, and wherein the vector further comprises a nucleic acid that encodes a non-spumavirus peptide, polypeptide, or protein. Thus, the present invention provides compositions comprising live replicating spumavirus vectors.

11. Also provided by the present invention is a method of treating a subject with a condition, wherein the condition can be a viral infection, bacterial infection, parasitic infection, proliferative disorder (eg. cancer), or a condition associated with a genetic or autoimmune disorder; comprising administering to the subject a live replicating viral vector, wherein the immunizing construct is specific for the condition.

12. Also provided by the present invention is a method of preventing a condition in a subject, wherein the condition can be a viral infection, bacterial infection, parasitic infection, proliferative disorder, or a condition associated with a genetic or autoimmune disorder; comprising administering to the subject a live replicating viral vector, wherein the antigen-encoding nucleic acid is specific for the condition.

13. Also provided are methods of using the present vector for making models and using models to study diseases and potential treatments, as well as the models themselves.

### BRIEF DESCRIPTION OF THE DRAWINGS

14. The accompanying drawings, which are incorporated in and constitute a part of this specification, illustrate several embodiments of the invention and together with the description, serve to explain the principles of the invention.

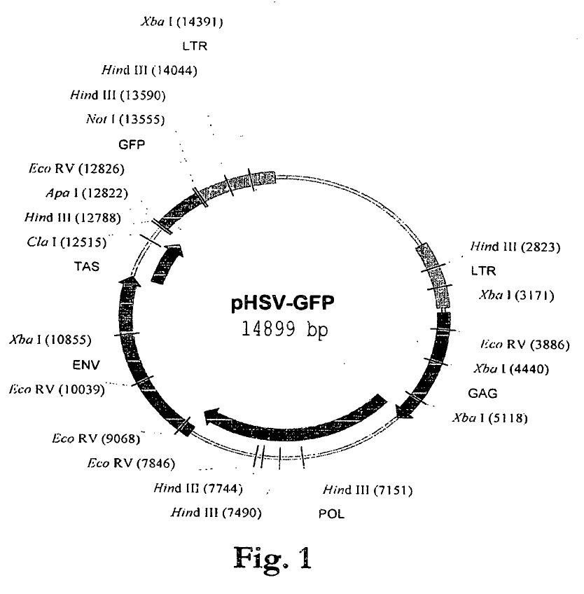

15. FIG. 1 shows a map of the pHSV vector detailing the location of the spumavirus env, pol, gag, and bel-1 (tas) genes. The map also indicates the presence of LTR flanking the coding region for the spumavirus genes and the presence and location of ApaI and NotI restriction sites.

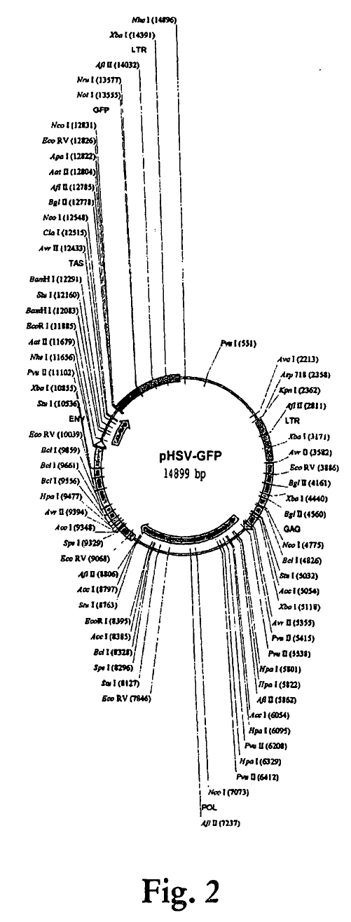

16. FIG. 2 shows a detailed restriction map of the pHSV vector. The sequence of the vector is provided in SEQ ID NO: 1.

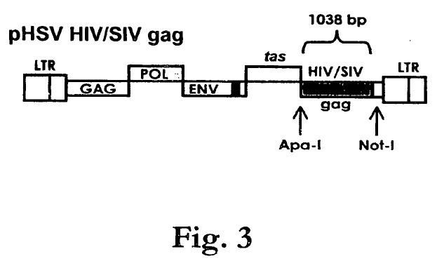

17. FIG. 3 shows a linear map of pHSV with the p 17/24 sequence incorporated in the vector to generate a pHSV-HIV-gag p17/24 vector. This shows the location of p17/24 within the vector.

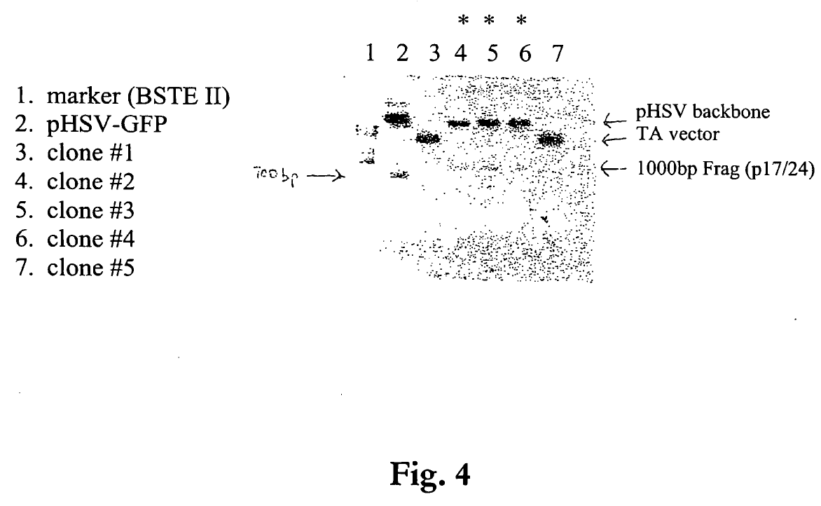

18. FIG. 4 shows that several TA-p17/24 clones have the desired length fragment when cut with the ApaI and NotI restriction enzymes. Shown is a 1% Acrylamide gel of purified clones resulting the transfection of p17/24 into the TA cloning vector (pCR2.1). Lane 1 is the marker BSTE II. Lane 2 is an empty pHSV vector used as a negative control. Lanes 3-7 are clones 1-5.

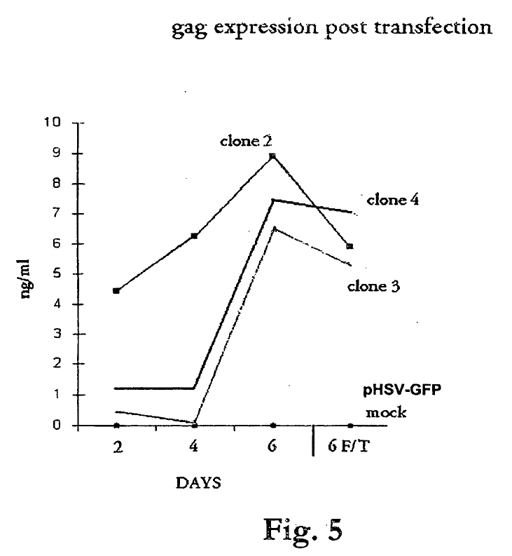

19. FIG. 5 shows that p24 expression can be measured by ELISA in clones 2, 3, and 4 at days 2, 4, and 6 days post infection of BHK cells. Samples were also measured for p24 expression 6 days post-infection following freeze/thaw (F/T) of the clones.

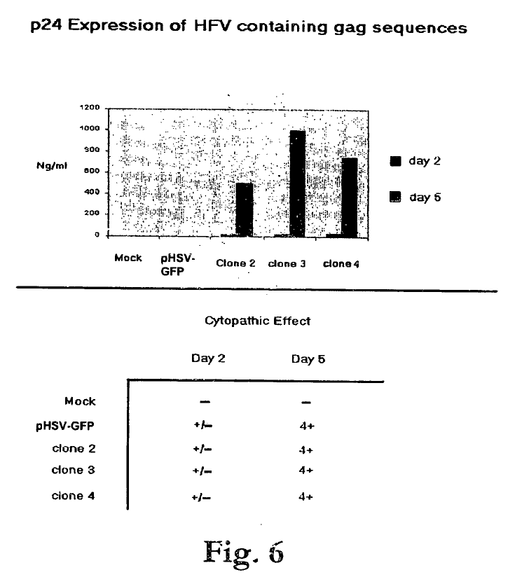

20. FIG. 6 shows that p24 expression is pronounced 5 days post infection in clone 2, clone 3 and clone 4.

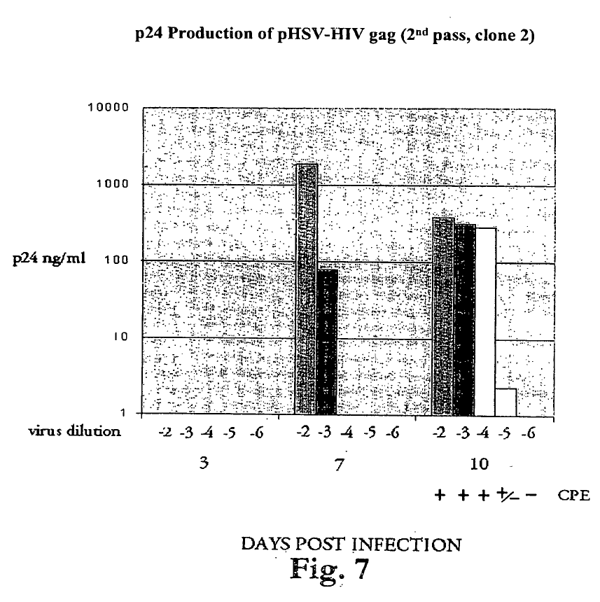

21. FIG. 7 shows that expression levels of p24 are maintained at high levels even on subsequent passages. Second pass clone 2 is diluted 10-fold and dilution was used to infect BHK cells. Supernatants were removed at 3, 7, and 10 days post infection and measured via ELISA for p24 expression.

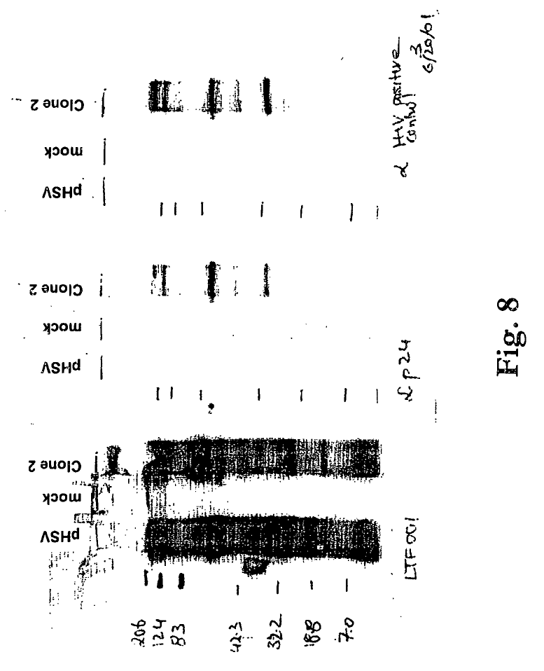

22. FIG. 8 shows Western blot analysis of clone 2. Lane 1 is empty pHSV vector, lane 2 is mock infected BHK cells, and lane 3 is BHK cells infected with clone 2. Samples were probed with LTF001, anti-p24, or anti-HIV serum and developed.

### DETAILED DESCRIPTION

22. The present invention may be understood more readily by reference to the following detailed description of preferred embodiments of the invention and the Examples included therein and to the Figures and their previous and following description.

23. Before the present compounds, compositions, and/or methods are disclosed and described, it is to be understood that this invention is not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Definitions 24. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

25. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

26. In this specification and in the claims which follow, reference will be made to a number of terms which shall be defined to have the following meanings:

27. “Optional” or “optionally” means that the subsequently described event or circumstance may or may not occur, and that the description includes instances where said event or circumstance occurs and instances where it does not.

Vectors

28. Disclosed are live replicating human spumavirus vectors suitable for human use comprising an immunizing construct, wherein the immunizing construct is inserted in the bet gene. The disclosed immunizing construct can be an antigen-encoding nucleic acid.

29. Where reference is made to “antigen”-encoding nucleic acid, it is understood that in the context of the invention antigens encoded by the antigen-encoding nucleic acid can include but are not limited to immunogenic or non-immunogenic peptides, polypeptides, proteins, enzymes, cytokines. These antigens can be non-human exogenous antigenic sequences from viruses, bacteria, or parasites. The antigens can also be antigenic endogenous human or human derived sequences from a condition such as a cancer. Also, peptides encoded by the antigen-encoding nucleic acid can include non-antigenic sequences for the purposes of gene therapy.

30. Also disclosed are vectors of the invention, wherein the vector is pHSV or pHSV-GFP. Though at any one time reference may only be made to one of the vectors, it is an embodiment of the invention that both vectors may be used interchangeably and with equivalent results. Accordingly terms referring to either vector should be understood to refer to both vectors. Similarly, reference throughout this disclosure is given to the pHSV vector containing an HIV antigen-encoding nucleic acid. It is understood that this construct may be referred to as pFOV-gag, pFOV7-gag, pFOV-7-gag, pFOV7-p17/24, pFOV-7-p17/24, pFOV-p17/24, pFOV-7GFP-gag, pFOV-7-GFP-gag, pFOV-7GFP-p17/24, pFOV-7-GFP-p17/24, pFOV-7 HIV-gag p17/24, pHSV-gag, pHSV-p17/24, pHSV-GFP-gag, pHSV-GFP-p17/24 or pHSV-HIV-gag-p17/24. A vector of the invention, can have the sequence of pHSV-HIV-GAG p17/24. Thus, herein disclosed is a pHSV vector of the invention comprising SEQ ID NO: 1, wherein an antigenic construct comprising the gag gene of HIV- 1 (SEQ ID NO: 2) has been inserted into the sequence at the unique ApaI site at 12816 and the unique Not I site at 13552. Said insertion resulting in the excision of nucleotides 12817-13551 of the vector and therefore partially replacing the bet gene of pHSV with SEQ ID NO: 2 (SEQ ID NO: 8). Therefore, also disclosed are vectors of the invention, wherein the antigen-encoding nucleic acid is HIV-GAG p17/p24. Herein, p17/24 and p41 are used synonymously and are intended to refer to DNA encoding Gag or an antigenic fragment of Gag. It is also herein contemplated that other genes from HIV may be used as the antigen-encoding nucleic acid in the vector of the invention. Such genes can include the env and pol genes of HIV as well as vpu, vif and nef genes.

31. Also disclosed are vectors of the invention, wherein the vector has the sequence of pHSV, which is defined by the restriction map shown in FIGS. 1, 2, and 3. It is an embodiment of the present invention that modifications can be made to the pHSV vector to incorporate additional unique restriction sites throughout the vector including but not limited to within and around the bel-2(bet), pol, gag, env, or bel-1 genes. It is also understood that such modifications may provide additional features including but not limited to reporter function, resistance to a pharmaceutical agent, or enhancers. Related, but distinct vectors are disclosed in U.S. Pat. No. 5,646,032 which is incorporated in its entirety herein by reference.

32. It is well-known in the art that vaccinations can be used prophylacticly for the prevention of infections as well as therapeutically for the treatment of ongoing conditions. Such infections or conditions can be but are not limited to viral infections. Thus, also disclosed are vectors of the invention, wherein the antigen-encoding nucleic acid is an antigen from a virus. The viral antigen can be selected from the group of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2, and Simian Immunodeficiency virus (SIV). Also disclosed are vectors of the invention, wherein the antigen-encoding nucleic acid is SIV-GAG. The art is repleat with examples of viral antigens whose sequences and methods of obtaining them are well known. Thus, a pHSV vector expressing these antigens is within the scope of the present invention.

33. Vaccinations are also known for the prevention of bacterial infections. Additionally, anitbiotics are well-known in the art for the treatment of various bacterial infections. Herein contemplated and disclosed are vectors of the invention, wherein the antigen-encoding nucleic acid is an antigen from a bacterium. The bacterial antigen can be selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Ricketisial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. The art is repleat with examples of bacterial antigens whose sequences and methods of obtaining them are well known. Thus, a pHSV vector expressing these antigens is within the scope of the present invention.

34. The vectors of the invention are not limited to bacteria and viruses. Also disclosed are vectors of the invention, wherein the antigen-encoding nucleic acid is an antigen from a parasite. The parasitic antigen can be selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica. The art is repleat with examples of parasitic antigens whose sequences and methods of obtaining them are well known. Thus, a pHSV vector expressing these antigens is within the scope of the present invention.

35. The treatment of various forms of cancer is a major concern for millions of people worldwide and the focus of much of medical research. Herein contemplated are methods of treating a cancer comprising administering to a subject the vector of the invention. Therefore, also disclosed are vectors of the invention, wherein the antigen-encoding nucleic acid is a tumor antigen. The tumor antigen can be selected from the list consisting of human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, and melanoma gangliosides. Each of these antigens is known and has a coding sequence that is publically available and well-known in the art. Further cancer antigens, whether later discovered or presently know can be expressed by the present pHSV vector as described herein.

36. There are instances wherein it is advantageous to administer the vector of the invention in a pharmaceutical composition that comprises other vaccines. Pharamceutical compositions comprising multiple vaccines can be for therapeutic or prophylactic purposes. An example Examples of such a composition is other vaccines include the mumps, measles, rubella (MMR) vaccine, and vaccines against M. tuberculosis, M. bovis, M. bovis strain BCG, BCG substrains, M. avium, M. intracellulare, M. africanum, M. kansasii, M. marinum, M. ulcerans, M. avium subspecies puratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species . Specifically contemplated and disclosed are pharmaceutical compositions comprising the vector of the invention and one or more additional vaccines. Also disclosed and herein contemplated are instances in which the vector of the invention comprises more than one antigen-encoding nucleic acid. In such a situation the vector of the invention will produce each antigen encoded in the vector as a separate antigen.

37. There are instances in which a vector of the invention alone may not be suitable for a given purpose (eg., A kit designed to screen potential drugs for the treatment of a condition such kit, intended for use in laboratories without the capabilities to transfect a cell-line with the vector). In such cases, cells previously transfected with the vector of the invention are needed. Thus, also disclosed are cells comprising the vector of the invention.

38. In an embodiment of the invention the antigen-encoding nucleic acid can encode a non-antigenic sequence of DNA. This sequence can provide a functional copy of a disrupted, mutated, disregulated or deleted gene. Examples of nucleic acids encoding proteins that play a role in genetic disorders are known in the literature relating to genetic disorders which is incorporated herein by reference. Methods of making these cells are described and exemplified herein and in the art.

39. The ability to detect the presence of a construct can be a desireable feature of any vector. As such, vectors arc often contain a marker to show that the construct of interest has been delivered to the subject (eg. a cell), and once delivered, is being expressed. A marker can take the form of a gene that is detectable when expressed. Thus, also disclosed are vectors of the invention further comprising a reporter gene. One example of a reporter gene is green fluorescence protein (GFP).

Compositions

40. The invention includes a composition comprising a vector of the invention. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular spumavirus vector or antigen-encoding nucleic acid (e.g., p17, p24, p17/24, gag, gp120) is disclosed and discussed and a number of modifications that can be made to a number of molecules including the spumavirus vector or antigen-encoding nucleic acid (e.g., p17, p24, p17/24, gag, gp120) are discussed, specifically contemplated is each and every combination and permutation of spumavirus vector and antigen-encoding nucleic acid (e.g., p17, p24, p17/24, gag, gp120) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods. Often therapeutic agents and vaccines are administered in formulations or combinations that incorporate other therapeutic modalities or necessary components for the purposes of time release, delivery, or augmentation of a response. It is understood and herein contemplated that the disclosed vectors and exogenous nucleic acid of the invention can be combined and administered with any such modality or component.

Expression Systems

41. The nucleic acid vectors of the invention that are delivered to cells typically contain expression controlling systems for controlling the expression of heterologous/exogenous nucleic acid. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Viral Promoters and Enhancers

42. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus, cytomegalovirus and most preferably spumavirus, or from heterologous mammalian promoters, e.g. beta actin promoter. Each of these promoters in known and has a sequence that is publicly available. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein. Methods of functionally linking promoters with coding sequences are well-known.

43. Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

44. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

45. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

46. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

47. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

Markers

48. The viral vectors can include nucleic acid sequence encoding a marker product (reporter gene). This marker product is used to determine if the gene has been delivered to the cell and once delivered, is being expressed. Marker genes can be but are not limited to the E. Coli lacZ gene, which encodes β-galactosidase, and green fluorescent protein.

49. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

50. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Treatment and Prevention Methods

51. By “treating” is meant an improvement in the disease state (i.e., viral infection, bacterial infection, parasitic infection, cancer, genetic disorder, or autoimmune disease) is observed and/or detected upon administration of a substance of the present invention to a subject. Treatment can range from a positive change in a symptom or symptoms of the disease to complete amelioration of the viral infection, bacterial infection, parasitic infection, or cancer (e.g., reduction in severity or intensity of disease, alteration of clinical parameters indicative of the subject's condition, relief of discomfort or increased or enhanced function), as detected by art-known techniques. The methods of the present invention can be utilized to treat an established viral infection, bacterial infection, parasitic infection, or cancer. One of skill in the art would recognize that viral infection, bacterial infection, parasitic infection, or cancer refer to conditions characterized by the presence of a foreign pathogen or abnormal cell growth. Clinical symptoms will depend on the particular condition and are easily recognizable by those skilled in the art of treating the specific condition. Treatment methods can include, but are not limited to therapeutic vaccinations. Thus, Disclosed are methods of treating a subject with a condition comprising administering to the vector of the invention.

52. Also disclosed are methods of the invention, wherein the condition being treated is a viral infection. The viral infection can be selected from the list of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

53. Also disclosed are methods of the invention, wherein the antigen-encoding nucleic acid is an antigen from a virus. The viral antigen can be selected from the group of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

54. Also disclosed are methods of the invention, wherein the condition being treated is a bacterial infection. The bacterial infection can be selected from the list of bacterium consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae. Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

55. Also disclosed are methods of the invention, wherein the antigen-encoding nucleic acid is an antigen from a bacterium. The bacterial antigen can be selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species; Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

56. Also disclosed are methods of the invention, wherein the condition being treated is a parasitic infection. The parasitic infection can be selected from the list of parasites consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

57. Also disclosed are methods of the invention, wherein the antigen-encoding nucleic acid is an antigen from a parasite. The parasitic antigen can be selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

58. Also disclosed are methods of the invention, wherein the condition being treated is cancer.

59. The disclosed vectors and vector containing compositions can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

60. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

61. Also disclosed are methods of the invention, wherein the antigen-encoding nucleic acid is a tumor antigen. The tumor antigen can be selected from the list consisting of human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp110/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, and melanoma gangliosides.

62. Disclosed are methods of treating a condition in a subject comprising administering to the subject the vector of the invention, wherein the condition is due to a mutated, disregulated, disrupted, or deleted gene; autoimmunity; or inflammatory diseases.

63. Disclosed are methods of treating a condition in a subject, wherein the condition can be selected from list consisting of cystic fibrosis, asthma, multiple sclerosis, muscular dystrophy, diabetes, tay-sachs, spinobifida, cerebral palsy, parkinson's disease, lou gehrigg disease, alzhcimer's, systemic lupus erythamatosis, hemophelia, Addsion's disease, Cushing's disease.

64. By “preventing” is meant that after administration of a substance of the present invention to a subject, the subject does not develop the symptoms of the viral, bacterial, or parasitic infection, and/or does not develop the viral, bacterial, or parasitic infection. “Preventing” or “prevention” can also refer to the ultimate reduction of an infection, condition, or symptoms of an infection, or condition relative to infections or conditions in subjects that do not receive the substance. Methods of prevention can include, but are not limited to profilactic vaccination. As such, disclosed are methods of preventing an infection in a subject comprising administering to the subject the vector of the invention.

65. Also disclosed are methods of the invention, wherein the infection prevented is a viral infection. The viral infection can be selected from the list of viruses consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

66. Also disclosed are methods of the invention, wherein the antigen-encoding nucleic acid is an antigen from a virus. The viral antigen can be selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-l, Hantavirus, Rubella virus, Simian Immunodeficiency virus, Human Immunodeficiency virus type-1, and Human Immunodeficiency virus type-2.

67. Also disclosed are methods of the invention, wherein the infection prevented is a bacterial infection. The bacterial infection can be selected from the list of bacterium consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

68. Also disclosed are methods of the invention, wherein the antigen-encoding nucleic acid is an antigen from a bacterium. The bacterial antigen can be selected from the group consisting of M. tuberculosis, M. bovis, M. bovis strain BCG, BCG substrains, M. avium, M. intracellulare, M. africanum, M. kansasii, M. marinun, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

69. Also disclosed are methods of the invention, wherein the infection prevented is a parasitic infection. The parasitic infection can be selected from the list of parasites consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

70. Also disclosed are methods of the invention, wherein the antigen-encoding nucleic acid is an antigen from a parasite. The parasitic antigen can be selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

71. Also disclosed are methods of the invention, wherein the subject is a horse, cow, pig, dog, car, mouse, monkey, human, or a cell isolated from such an animal.

Delivery of the Compositions to Cells

72. There are a number of compositions and methods which can be used to deliver nucleic acids to cells, either in vitro or in vivo. For example, the nucleic acids can be delivered through a number of direct delivery systems such as, electroporation, lipofection, calcium phosphate precipitation, and the present viral vectors. Appropriate means for transfection, including the present viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

73. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

74. As used herein, viral vectors such as pHSV are agents that transport the disclosed antigen-encoding nucleic acids, such as p17/24 into the cell without degradation and include a promoter yielding expression of the gene in the cells into which it is delivered. Retroviral vectors especially spumavirus vectors are able to carry a larger genetic payload, i.e., a transgene or marker gene, than other viral vectors. A preferred embodiment is a viral vector which has been engineered so as to induce the immune response of the host organism, elicited by the peptides encoded on the vector.

75. Viral vectors can have higher transaction abilities (ability to introduce genes) than chemical or physical methods to introduce genes into cells. Typically, viral vectors contain, nonstructural and structural genes, a polymerase, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry large fragments of foreign genetic material. The necessary functions of the removed early genes are typically supplied by cell lines which have been engineered to express the gene products of the early genes in trans.

76. Spumaviruses are retroviruses. A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp.229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference. Although the present spumavirus vector is unique, the methods described for using other types of viral vectors can be useful in certain contexts. See for example U.S. Pat. No. 5,646,032, which is incorporated herein for its teaching of those methods.

77. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. The removal of the gag, pol, and env genes allows for large fragments of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

78. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

In Vivo/Ex Vivo

79. As described herein, the vector-containing compositions can be administered in a pharmaceutically acceptable carrier and can be delivered to the subject [symbol] s cells in vivo and/or ex vivo by a variety of mechanisms well known in the art (e.g., uptake of naked DNA, liposome fusion, intramuscular injection of DNA via a gene gun, endocytosis and the like).

80. If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

Nucleic Acid Delivery

81. In the methods described above which include the administration and uptake of exogenous DNA into the cells of a subject (i.e., gene transduction or transfection), the nucleic acids of the present invention can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art.

82. As one example, if the antigen-encoding nucleic acid of the invention is delivered to the cells of a subject in a spumavirus vector, the dosage for administration of spumavirus to humans can range from about 10⁷ to 10⁹ plaque forming units (pfu) per injection but can be as high as 10¹⁰ to 10¹² pfu per injection. In some cases lower dosages (eg., 10³, 10⁴, 10⁵, and 10⁶ pfu) can be effective. A subject can receive a single injection, or, if additional injections are necessary, they can be repeated at two, four, six month intervals (any intervening time intervals or other appropriate time intervals, as determined by the skilled practitioner) for an indefinite period and/or until the efficacy of the treatment has been established.

83. Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of pharmacy (19th ed.) ed. A. R. Gennaro, Mack Publishing Company, Easton, Pa. 1995.

Delivery of Pharmaceutical Products

84. The vector or vector-containing compositions can be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

85. The compositions may be administered orally, parenterally (e.g., intravenously), by intramuscular injection, by intraperitoneal injection, subcutaneously, transdermally, extracorporeally, topically, gene gun or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

86. The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399409 (1991)).

Pharmaceutically Acceptable Carriers

87. The compositions, can be used therapeutically in combination with a pharmaceutically acceptable carrier.

88. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

89. Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

90. The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compositions can be administered intravenously, intraperitoneally, intramuscularly, subcutaneously, intracavity, by gene gun, or transdermally.

91. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

92. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

93. Compositions for oral administration include powders or granules, suspensions or solutions in water or non-aqueous media, capsules, sachets, or tablets. Thickeners, flavorings, diluents, emulsifiers, dispersing aids or binders may be desirable.

94. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Therapeutic Uses

95. The substances of the present invention can be delivered at effective amounts or concentrations. An effective concentration or amount of a substance is one that results in treatment or prevention of the condition (e.g. HIV or AIDS). One skilled in the art would know how to determine an effective concentration or amount according to methods known in the art, as well as provided herein. One of skill in the art can utilize in vitro assays to optimize the in vivo dosage of a particular substance, including concentration and time course of administration.

96. Herein, immune modulatory substances are described. As defined immune modulation refers to any change in the immune response. This includes but is not limited to increase or decreases in the number of antigen specific plasma cells, memory B cells, memory T cells, activated CD8 T cells, cytokine production, and cytolitic killing or the maintenance of homeostatic levels of the same when the responses would otherwise be changing. The dosage ranges for the administration of the substances are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

97. For example, to evaluate the efficacy of treatment of humans with a condition, such as for example, HIV, with a substance that modulates normal immune responses to HIV, the following studies can be performed. Patients with active infection can be selected. Drug efficacy can be monitored via viral titer, antibody ELISA or ELISPOT and CD4 T cell count. Patients can be randomized to two different protocols. In one protocol, subjects can remain on initial medication and in the second protocol, subjects can have their medication tapered after receiving the substance that modulates immune responses.

98. In one embodiment, treatment can consist of either a single dosage of 0.3 mg to 0.6 mg/animal of the vector expressing a substance that treats or prevents the condition. Additionally, dosage as low as 30 μg/animal to 300 μg/animal can be used with intramuscular injection of infecftious DNA. In one example, a DNA immunization comprising 0.3 mg of the pFOV-gag vector is administered to mouse via gene gun. After two weeks the subject is monitored using conventional assays to assess a generated immune response to p 17/24 in the form of antibody production and antigen specific T cells. Having generated a response, the mouse is then challenged with an infectious dose of HIV. The mouse is then monitored for disease progression and clinical symptoms associated with HIV. Alternatively, a mouse can be infected with HIV and then given a therapeutic dose of the pFOV-gag. The animal is monitored for changes in the viral lode, clinical progression of symptoms, as well as immune responses in comparison to non treated control animals. In a further example, a DNA immunization comprising 0.05 μg (50 μg) of the pFOV-gag vector is administered to mouse via intrauscular injection of infectious DNA. After two weeks the subject is monitored using conventional assays to assess a generated immune response to p17/24 in the form of antibody production and antigen specific T cells. Having generated a response, the mouse is then challenged with an infectious dose of HIV. The mouse is then monitored for disease progression and clinical symptoms associated with HIV. Alternatively, a mouse can be infected with HIV and then given a therapeutic dose of the pFOV-gag. The animal is monitored for changes in the viral lode, clinical progression of symptoms, as well as immune responses in comparison to non treated control animals. The art of determining dosage for an animal based on size is well known. It is understood that a skilled artisan would be able to determine the proper dosage of a substance for an animal based on the dosage of the same substance administered to another animal of similar or different size. For example, wheras a 200g mouse would receive a 50 μg dose of the immunization, a 150 lb (68 kg) human would be administered an immunization comprising about 17 mg of the vector.

99. Disclosed are methods of detecting the expression of the vector of the invention comprising using a first antibody to the antigen to measure protein expression in a quantitative or qualitative way, further comprising detecting the first antibody directly via a calorimetric measurement produced through the use of a substrate and a conjugated antibody or indirectly via a firstantibody to the antigen which in turn is bound by a second antibody which is conjugated and will result in a colorimetric measurement when combined with a substrate.

100. Also disclosed are methods of the invention, wherein the antigen is detected by placing an aliquot of the vector of the invention in a lane on a gel and probing the gel for the antigen.

101. Disclosed are methods of detecting the expression of the vector of the invention comprising using a fluorescently labeled first antibody specific for the antigen and visualizing the antigen using a flow cytometer or fluorescence microscope.

102. Also disclosed are methods of the invention, wherein the first antibody is not flourescently labeled, but a target for a second antibody with a fluorescent label.

103. Disclosed are methods of detecting the expression of the vector of claim the invention comprising using cytolitic killing assay to assess activity.

104. Disclosed are methods of detecting the vector of the invention further comprising obtaining a sample from a subject comprising a tissue biopsy or removal of blood or bone marrow.

Screening Methods

105. Also provided by the present invention is a method of screening a substance for effectiveness in treating or reducing the severity of the condition (e.g., HIV infection) comprising: a) obtaining an animal having the condition or characteristic (e.g., symptom) of the condition; b) administering the substance to an animal having one or more characteristics of the condition; and assaying the animal for an effect on the condition, thereby identifying a substance effective in reducing the condition.

106. The ability of a substance to reduce the severity of a condition can be determined by evaluating the histological and clinical manifestations, of the animal with condition before and after administration of the substance of interest and quantitating the amount of reduction of the condition.

107. The animal in which the condition or characteristic (e.g., symptom) of the condition is produced can be any mammal and can include but is not limited to mouse, rat, guinea pig, hamster, rabbit, cat, dog, goat, monkey, and chimpanzee. The condition or characteristic (e.g., symptom) of the condition can be produced in the animal by any method known in the art. For example, HIV can be produced by introducing into the animal (eg., chimpanzee infected with HIV or rhesus macaques or nemestrina macaques infected with an HIV-1 env on an SIV backbone. Pullium, J K, et. al., J. Infectious Dis. 183:1023, 2001) an infectious amount of HIV.

108. The present invention also provides a method of screening for a substance effective in preventing the condition (e.g., HIV infection) comprising: a) administering the substance to an animal susceptible to the condition; b) subjecting the animal to treatment that will induce the condition or characteristic (e.g., symptom) of the condition; and c) assaying cells from the animal for an change in immune responses as compared to an the immune responses in a control animal having the condition in the absence of the substance identifies a substance that is effective in preventing the condition.

109. For example, the methods of measuring the amount of p17/24 or other HIV antigen in an animal include, but are not limited to, ELISA, PCR, FACS analysis, reverse-transcriptase-polymerase chain reaction and ELISPOT, Northern blots, Southern blots, and Western blots.

110. A model for use in screening for substances effective in treating or preventing a disease comprising an animal capable of manifesting a characteristic of the disease is provided, wherein the animal has been administered the vector of the invention.

111. A method of making the model of the invention comprising obtaining administering to an animal capable of manifesting a characteristic of the disease and administering to said animal the vector of claim 1 a vector of the invention which encodes an antigen associate with the disease.

112. A method of screening for a substance effective in treating a disease associated with an immunizing construct comprising: a) administering the substance to the model of the invention; and b) assaying for an change in the course of the disease as compared to an the course of the disease in a control subject; an improvement in the course of the disease in the presence of the substance identifies a substance that is effective in treating the disease.

113. A method of screening for a substance effective in preventing a disease associated with an immunizing construct comprising: a) administering a the vector of the invention to a subject; b) subjecting the subject to treatment that will induce the disease or characteristic (e.g., symptom) of the disease; and c) assaying for an change in the course of the disease as compared to an the course of the disease in a control subject; an improvement in the course of the disease in the presence of the substance identifies a substance that is effective in preventing the disease.

114. A method of screening for a substance effective in treating a disease associated with an immunizing construct comprising: a) subjecting the subject to treatment that will induce the disease or characteristic (e.g., symptom) of the disease; b) administering a the vector of the invention to a subject; and c) assaying for an change in the course of the disease as compared to an the course of the disease in a control subject; an improvement in the course of the disease in the presence of the substance identifies a substance that is effective in treating the disease.

Sequence Similarities

115. It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

116. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

117. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

118. The same types of homology can be obtained for nucleic acids by for example the algorithms disclosed in Zuker, M. Science 244:48-52, 1989, Jaeger et al. Proc. Natl. Acad. Sci. USA 86:7706-7710,1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

119. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Nucleic Acids

120. There are a variety of molecules disclosed herein that are nucleic acid based, including for example the nucleic acids that encode, for example HIV-1 gag, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

Sequences

121. There are a variety of sequences related to the gag gene that are publicly available (eg., Genbank Accession Number: L03707), these sequences and others are herein incorporated by reference in their entireties as well as for individual subsequences contained therein.

122. One particular sequence set forth in gag that is publicly available and having Genbank accession number L03707 is used herein, as an example, to exemplify the disclosed compositions and methods. It is understood that the description related to this sequence is applicable to any sequence related to gag unless specifically indicated otherwise. Those of skill in the art understand how to resolve sequence discrepancies and differences and to adjust the compositions and methods relating to a particular sequence to other related sequences (i.e. sequences of gag). Primers and/or probes can be designed for any gag sequence given the information disclosed herein and known in the art.

Peptides/Protein Variants

123. There are numerous variants of protein antigens that are antigenic. For example, there are variants of the gag protein and protein that are known and herein contemplated. In addition to the known functional HIV-1 gag strain variants there are derivatives of the gag proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| Alanine | Ala, A |
| Allosoleucine | AIle |
| Arginine | Arg, R |
| Asparagine | Asn, N |
| aspartic acid | Asp, D |
| Cysteine | Cys, C |
| glutamic acid | Glu, E |
| Glutamine | Gln, Q |
| Glycine | Gly, G |
| Histidine | His, H |
| Isolelucine | Ile, I |
| Leucine | Leu, L |
| Lysine | Lys, K |
| Phenylalanine | Phe, F |
| Proline | Pro, P |
| pyroglutamic acidp | Glu |
| Serine | Ser, S |
| Threonine | Thr, T |
| Tyrosine | Tyr, Y |
| Tryptophan | Trp, W |
| Valine | Val, V |

TABLE 2
Amino Acid Substitutions
Original Residue Exemplary Conservative Substitutions,
others are known in the art.

Ala; Ser
Arg; Lys, gln
Asn; Gln; his
Asp; Glu
Cys; Ser
Gln; Asn, lys
Glu; Asp
Gly; Pro
His; Asn; gln
Ile; Leu; val
Leu; Ile; val
Lys; Arg; gln;
Met; Leu; ile
Phe; Met; leu; tyr
Ser; Thr
Thr; Ser
Trp; Tyr
Tyr; Trp; phe
Val; Ile; leu

124. Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

125. The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, lie, Leu; Asp, Glu; Asn, Gln; Ser, Thr; Lys, Arg; and Phe, Tyr. Such conservatively substituted variations of each explicitly disclosed sequence are included within the antigenic polypeptides encoded and expressed by the vectors provided herein.

126. Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

127. Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

128. It is understood that one way to define the variants and derivatives of the disclosed proteins herein is through defining the variants and derivatives in terms of homology/identity to specific known sequences. For example, SEQ ID NO:3 sets forth a particular sequence of a gag protein. Specifically disclosed are variants of these and other proteins herein disclosed which have at least, 70% or 75% or 80% or 85% or 90% or 95% homology to the stated sequence. It is understood that those of skill in the art will recognize variants of a disclosed protein as being a variant. Particularly, there are numerous naturally occurring variants of viral (e.g., HIV) antigens. For example, it is understood that those of skill in the art would recognize that the particular gag protein disclosed in SEQ ID NO: 4 is a gag protein from a different HIV-1 isolate from the HIV-1 gag protein in SEQ ID NO: 3 and therefore a variant that can be used with equivalent results. Those of skill in the art readily understand how to determine the homology of two proteins. For example, the homology can be calculated after aligning the two sequences so that the homology is at its highest level.

129. Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. 130. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol 183:281-306, 1989 which are herein incorporated by reference for at least material related to nucleic acid alignment.

131. It is understood that the description of conservative mutations and homology can be combined together in any combination, such as embodiments that have at least 70% homology to a particular sequence wherein the variants are conservative mutations.

132. As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 3 is set forth in SEQ ID NO: 2. In addition, for example, a disclosed conservative derivative of SEQ ID NO: 4 is shown in SEQ ID NO: 5, where the isoleucine (I) at position 19 is changed to a valine (V). It is understood that for this mutation all of the nucleic acid sequences that encode this particular derivative of the gag gene of the LAI strain of HIV-1 are also disclosed. It is also understood that while no amino acid sequence indicates what particular DNA sequence encodes that protein within an organism, where particular variants of a disclosed protein are disclosed herein, the known nucleic acid sequence that encodes that protein in the particular gag from which that protein arises is also known and herein disclosed and described.

Computer Readable Mediums

133. It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Methods of Making the Compositions

134. The compositions disclosed herein and the compositions necessary to perform the disclosed methods can be made using any method known to those of skill in the art for that particular reagent or compound unless otherwise specifically noted.

Nucleic Acid Synthesis

135. For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

136. Disclosed are cells produced by the process of transforming the cell with any of the disclosed nucleic acids. Disclosed are cells produced by the process of transforming the cell with any of the non-naturally occurring disclosed nucleic acids.

137. Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the disclosed non-natural nucleic acids.

138. Disclosed are animals produced by the process of transfecting a cell within the animal with any of the nucleic acid molecules disclosed herein. Disclosed are animals produced by the process of transfecting a cell within the animal with any of the nucleic acid molecules disclosed herein, wherein the animal is a mammal. Also disclosed are animals produced by the process of transfecting a cell within the animal with any of the nucleic acid molecules disclosed herein, wherein the mammal is mouse, rat, rabbit, cow, sheep, pig, or primate.

139. Also disclose are animals produced by the process of adding to the animal any of the cells disclosed herein.

Methods of Using the Compositions as Research Tools

140. The disclosed vectors, compositions and methods can be used for targeted gene disruption and modification in any animal that can undergo these events. Gene modification and gene disruption refer to the methods, techniques, and compositions involved in the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, for example, as described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

141. One of the preferred characteristics of performing homologous recombination in mammalian cells is that the cells should be able to be cultured, because the desired recombination events occur at a low frequency.

142. Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

143. Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence or section in which the reference is cited.

144. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

145. The following examples are put forth so as to provide those of ordinary skill in the art with a complete disclosure and description of how the vectors, compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

146. In order to obtain the p17/p24 fragment of the HIV-1 gag gene, pSX plasmid containing the gag gene was subjected to 30 cycles of PCR using forward Apa1-p17 5′ primer and reverse Not1-p24 3′ primer. PCR product was cloned into a TA cloning vector (pCR2.1 Invitrogen; Carlsbad, Calif.) and expanded. FIG. 1 shows a map of the empty pHSV vector. The vector contains the viral envelope gene env as well as its structural gene gag and viral polymerase gene pol. The vector also possesses a transactivator (TAS) gene bel-1 and 2 LTR that flank the coding region of the vector. An inspection of the vector SEQ (SEQ ID NO: 1) shows where various restriction sites are located on the vector. A complete listing of the sites is found in FIG. 2 and these sites are shown in SEQ ID NO: 1.

147. Inspection of the restriction map revealed ApaI and NotI unique restriction sites located around the BET (Bel-2) gene at 13522 and 12816 respectively. As such, the BET gene was chosen as the site for integration of the exogenous nucleic acid (FIG. 3). The p17/p24 were excised from the TA cloning vector using ApaI and NotI restriction enzymes. Similarly, the empty pHSV vector was cut with ApaI and NotI.

148. The p17/p24 segment of gag (789 (start of p17)—1876 (end of p24)) was ligated to pHSV and the pHSV-GAG was transformed into E. coli via electroporation. The bacteria was plated onto high Amp plates with the pHSV conferring ampR between the LTRs on the approximate 3 kb plamid. Seven colonies were chosen and checked for insertion and proper orientation. FIG. 4 shows that clone 2, 3,4 when cut with ApaI and NotI possessed a 1 kb fragment. This indicates that the p17/p24 fragment is intact and properly oriented.

149. Knowing that the construct was integrated into the vector, the next objective was to examine expression levels of the antigen. Integration is meaningless if the desired protein is not expressed. To rest expression of p17/p24, the loaded pHSV vector was transfected using lipofectamine into BHK cells and supernatants were collected at various time points post transfection. ELISA testing for p24 expression was conducted on the collected supernatants (FIG. 5).

150. Following transfection, clone 2 had clearly measurable p24 expression. The p24 expression level for clone 2 was increased at each time point after day 2, clones 3 and 4 showed no measurable expression of p24 until day 5 post transfection. Subsequent Elisa data revealed that 5 days post transfection clones 2, 3, and 4 could produce as much as 500 ng/ml, 1000 ng/ml, and 800ng/ml of p24 respectively (FIG. 6). At the same time in vitro cytopathology was assessed. Cells from the day 2 cultures had very little CPE. However, by day 5 CPE was readily observed. The presence of CPE shows that the p24 activity was associated with the replicating viral vector.

151. FIG. 7 shows p24 expression in clone 2 after 2^{nd} pass. Supernatants were collected at 3, 7 and 10 days after infection with diluted virus. At day 3 no p24 is observed. However, by day 7 1000 ng/ml of p24 is observed at the 10⁻² dilution, which is confirmed at the 10^{31 3} dilution with a measurement of 100 ng/ml. By day 10, the 10^{31 2} to 10^{31 4} dilutions all had approximately 200 ng/ml of p24. As such, day 7 undiluted virus would yield 10⁵ ng/ml p24 and at day 10 the secretion level ranges from 2×10⁴ to 2×10⁶ ng/ml of p24. This is an extremely high expression level and indicates likely success in vivo as this vector is resulting in expression comparable to the highest 25% of vectors.

152. As a verification that p24 Elisa data was actually measuring p17/p24 (p41) and not just p24, western blotting was performed (FIG. 8). Supernatants from BHK cells infected with clone 2 pHSV-gag, empty pHSV, or mock infected (infected with PBS) were run on a polyacrylamide gel and probed for p24, HIV, or LTF001 as a positive control. LTF001 antibody bound to all 3 lanes. However, bands were apparent only in the lane for clone 2 when either anti-p24 or anti-HIV was used as a probe. The resulting bands show the smallest band to be 41kd, which corresponds to the size of p17/p24.

153. Histological observations were made from in vitro culture of pHSV in BHK cells. Cells infected with pHSV-gag showed CPE; however, this CPE is not usually observed in vivo.

Methods

Cloning and PCR

154. The p17 and p24 segments of the LAI strain of HIV-1 gag were cloned into a pHSV spumavirus vector. Briefly, pSX plasmid containing the LAI strain HIC-1 gag gene was obtained from the HIV repository. The pSX plasmid was used as a template for PCR amplification and cloning of p17 and p24. A forward ApaI-p17 5′ primer TCC GGG CCC GGA ATG CCT ATA GTC CAG AAC ATC C (SEQ ID NO: 6) and a reverse NotI-p24 3′ primer GCG GCC GCG TTT TGA GAA CGA AAT ACC GG (SEQ ID NO: 7) were used to amplify p41. The amplified segment was cloned into a TA cloning vector (pCR2.1; Invitrogen Carlsbad, Calif.) and expanded. Restriction digest of the TA-p41 vector with ApaI and NotI removed p17/24 from the vector. The p41 segment was then inserted into pHSV within the bet (bel-2) gene.

Transformation

155. The pHSV vector and p17/24 antigen were ligated together with ligase for 16 hrs at 4 C. Following ligation, pHSV-gag was electroporated into E. coli and incubated for 1 hr at 37 C in 2xyT broth media. The samples are then plated on high Ampicillin plates and incubated overnight at 37 C. Colonies growing on the high Amp plates were selected and transferred to 2xyT broth media overnight at 37 C. Minipreps were performed on the overnight cultures. Plasmids were cut with ApaI and NotI and aliquots were run on 1% agarose gel.

ELISA and Western Blot

156. ELISA and Western blot were performed using techniques commonly used in the art.

### Example 2

157. Animal subjects can be used to screen the effectiveness of a pHSV vector and an antigen-encoding nucleic acid. Additionally, animal subjects may be used to study the vector-antigen combination's ability to prevent or treat a condition. The vector can also be used to induce a condition in an animal that is associate with a disease, and that animal can then be used to study the disease/condition or to study potential treatments for the disease/condition. Such conditions can be infections resulting from viruses, bacteria, or parasites; autoimmune reactions including inflammatory diseases, asthma, systemic lupus erythamatosis, muscular dystrophy, or multiple sclerosis, diabetes, tay-sachs, spinobifida, cerebral palsy, parkinson's disease, lou gehrigg disease, alzheimer's, hemophelia, Addsion's disease, Cushing's disease; or cancer. Animals can include but are not limited to mice, rats, pig, dog, monkey, chimpanzee, and human.

158. Mice are injected with a sub-immunizing dose of pHSV-gag. 0.3 mg/ animal of pHSV-gag are administered via gene gun. This same immunizing dose may be administered by an alternative route such as intramuscular, intravenous, intraperitoneal, intracranial, or subcutaneous. Mice are then monitored and starting at 15 days post immunization mice are bled every three days to day 30 post immunization. Blood samples are collected for serum extraction and for the isolation of peripheral blood lymphocytes (PBL). Serum samples can be used immediately for ELISA assays to look for antibody to the antigen encoded in the vector or for cytokine increases following immunization. Samples can also be frozen and stored at −80 C for testing at a later time.

159. Lypmhocytes are removed from whole blood and diluted in media (e.g. RPMI 1640, DMEM, MEM, or EMEM) with 10% serum. PBL are then stained for antigen specific T cells using antigen specific MHC class I and MHC class II tetramers to be visualized via flow cytometry. Simultaneously or separately PBL are stained with surface markers to look at activation characteristics of the cells and other surface phenotypes. Some examples of the antibodies used are CD3, CD4, CD8, CD11a (LFA-1), CD43 (1B11), CD44, CD45RA, CD45RO, CD62L (L-selectin), CD69 and Bcl-2. Additionally, PBL can be stained for the production of intracellular cytokines such as IFN-γ, IL-4, TNF-α, IL-2, and IL-10 following stimulation with antigen. Lymphocytes can also be used in an ELISPOT assay for cytokine production following stimulation with specific antigen or CTL assay to assess killing activity.

160. All of the stains and assays allow for the assessment of cell-mediated responses to the priming antigen. Tetramer staining allows for the enumeration of antigen specific T cells of a known specificity. This coupled with surface markers details the activation state of the antigen specific cells. Tetramer positive T cells (CD8+/CD3+, CD3+, or CD3+/CD4+) possess various surface markers that reveal the activation state of the cells. By multiplying percentages obtained from the analysis of the staining by the number of cells, the exact number of cells exhibiting any characteristic can be obtained. Exposure to an antigen creates cells that express high levels of CD11a, and CD44. Once these levels are increased on a cell, they will not decrease, and in conjunction with tetramer stains and other surface markers can lead to the identification of memory T cells. CD43 and CD69 both increase during activation, but decrease following clearance of the priming antigen. L-selecting will decrease on the surface of activated cells and increase over time to naive levels. Bcl-2 also provides a measure of the activation level of cells as activated cells will have decreased Bcl-2 levels relative to naive T cells indicating a susceptibility to apoptosis whereas memory cells will have increased levels of Bcl-2.

161. ELISPOT and intracellular cytokine stains allow the enumeration of cytokine secreting antigen-specific cells following stimulation. This can be paired with the number of tetramer positive cells to determine what portion, if not all, of the T cells are functional.

162. CTL assays determine the ability for antigen specific T cells to kill cells expressing the antigen. The assay reads on the cytolitic activity of T cells as determined by release of a chemiluminescent marker or radioactive material in labeled target cells.

163. Like the cell-mediated responses, humoral responses may be measured in harvested PBL. PBL maybe stained for anti-mouse IgG, CD45R (B220), PNA, and CD138 (syndecan-1) to look at the activation of B cells or the number of plasma cells. Plasma cells, the effector arm of the humoral immune system are syndecan-1+, and B220−. Memory B cells will be IgG+, B220+, and PNA+, the presence of PNA positive cells indicates transition through a germinal center reaction. Additionally, PBL may be used to enumerate the number of antigen specific plasma cells on a plasma cell elispot or the number of antigen specific B cells on a memory B cell assay.

164. ELISA data can be combined with the humoral immunity data from the PBL to assess the effect of the immunization on the mouse.

165. Mice that have measurable responses can be boosted and responses monitored. However, direct challenge is necessary as it indicates the ability to mount a protective immune response. Mice that do not have an immune response or mice that have an immune response that but need to be boosted can receive second and third immunizations by the same or different route than the priming immunization over the next few months.

166. As mice cannot be infected with a lentivirus, monkeys (e.g., rhesus macaques) are used for challenge experiments. Monkeys can receive a similar construct (pHSV-SIV-gag) which produces similar levels of gag in vitro. Monkeys are inoculated with the pHSV-SIV-gag DNA, and after several weeks, they can be challenged with an infectious SIV. The animals can then be observed for protection from infection or disease.

167. For a challenge, monkeys are given an infectious dose of an antigen (e.g. SIV) or stimulated in such a way as to induce a condition to be prevented. Challenged monkeys can be assessed in the same way as immunized mice with additional assays used to look at antigen load following challenge and gross pathological and histological assessments made from tissue biopsy or necropsy.

168. Monkeys can also be given an infectious dose of an antigen (e.g. SIV) or stimulated in such a way as to create a condition to be treated. Once the condition is established, monkeys are given a therapeutic dose of the immunizing antigen and monitored as to the effects on the condition. A successful therapy will not necessarily clear the condition or infection, but may slow or stop progression. The same assays to those used for prophylactic immunization are used to characterize the responses

Methods

Flow Cytometry

169. Cells are suspended at a concentration that is appropriate according to the antibody manufacturers instructions in FACS buffer (2% FCS (or BSA) in PBS (0.2% NaN₃ can also be added)). Antibodies (CD3, CD4, CD8, CD11a (LFA-1), CD43 (1B11), CD44, CD45R (B220), CD45RA, CD45RO, CD62L (L-selectin), CD69, CD138, PNA, anti-mouse IgG) are added to the cells according to the manufacturers instructions and incubated for 30 min at 4 C in the dark. Cells are washed 3× in FACS Buffer. 1 wash comprises centrifuging the cells at 800 rpm for 3 min to pellet the cells, removing the media, and resuspending the cells. After the third wash, cells are resuspended in 2% PFA in PBS. Staining is analyzed on a FACSCalibur flow cytometry instrument (Beckton-Dickenson) or other suitable cytometer.

Intracellular Cytokine Staining.

170. Cells are stimulated for 5 hrs in the presence of antigen and BFA (meninsen may be substituted for BFA) at 37 C (this stimulation is not needed for Bcl-2 staining). After the 5 hr incubation, cells are centrifuged and the media removed and resuspended in FACS buffer (2% FCS (or BSA) in PBS (0.2% NaN₃ can also be added)). Antibodies (CD3, CD4, CD8, CD11a (LFA-1), CD43 (1B11), CD44, CD45R (B220), CD45RA, CD45RO, CD62L (L-selectin), CD69, CD138, PNA, anti-mouse IgG) are added to the cells according to the manufacturers instructions and incubated for 30 min at 4 C in the dark. Cells are washed 3× in FACS Buffer. 1 wash comprises centrifuging the cells at 800 rpm for 3 min to pellet the cells, removing the media, and resuspending the cells. After the third wash, cells are resuspended in cytofix/cytoperm solution for 20 min at 4 C. Cells are then washed 3× in a permwash solution and resuspended at a concentration that is appropriate according to the antibody manufacturers instructions in permwash. Antibodies (IFN-γ, TNF-α, IL-2, IL-4, IL-10, and Bcl-2) are added to the cells according to the manufacturers instructions and incubated for 30 min at 4 C in the dark. Cells are washed 3× in PermWash and 2× in FACS Buffer. After the second FACS Buffer wash, cells are resuspended in 2% PFA in PBS. Staining is analyzed on a FACSCalibur flow cytometry instrument (Beckton-Dickenson) or other suitable cytometer. Intracellular kits are commercial available through (Beckton-Dickenson).

ELISA

171. 96-well ELISA plates are coated with antigen overnight at 4 C. Plates are blocked with a suitable blocking media containing 10% FCS for 1-2 hrs at RT. Plates are resuspended in a known volume of an ELISA diluent (e.g., PBS+10% FCS) and serum is added to the plate and incubated at RT for 1.5 hrs. Plates are washed with PBS+0.1% Tween 3× and 100 μl of antibodies (e.g., HRPO conjugated anti-mouse IgG) diluted 1:1000 in ELISA diluent is added to each well. Plates are incubated for 1.5 hrs at RT and then washed 3× with PBS+0.1% Tween. Plates are then coated with 100 μl of a chromagen substrate (e.g., o-phenyldiamine+3% H₂O₂ in citrate buffer (Sodium citrate in H₂O pH=5) and incubated for 1 hr in the dark. The reaction is stopped by adding 100 μl of 1N HCl. Plates are read in a ELISA plate reader.

Plasma Cell ELISPOT

172. 96-well filter (ELISPOT) plates are coated with antigen overnight. Plates are washed 1× in PBS-0.1% Tween and 3× in PBS. Plate are blocked 1-2 hrs with media+10% FCS. After blocking, media is removed and 100 μl of media are added to each well. Effector cells (cells containing lymphocytes from an immunized animal (e.g., PBL, splenocytes, hepatocytes, and bone marrow) are added to the plate and serial dilutions are made. Plates are incubated for 5 hrs at 37 C. After the 5 hr incubation, plates are washed 3× in PBS and 3× in PBS+0.1% Tween. Plates are then coated with 100 μl PBS+0.1% Tween+1% FCS and biotinylated Ab at 1/100 concentration overnight at 4 C. After incubation plates are washed 4× in PBS+0.1% Tween and incubated for 1 hr at RT in PBS+0.1% Tween+1% FCS containing HRP-avidinD at 1/1000. Plates are washed 3× with PBS and a suitable chromagen substrate is added (e.g., AEC). Plates are allowed to develop for 8-15 min and the reaction is stopped with washing the plates with water. Spots are visualized and counted using a dissection microscope or ELISPOT plate reader.

Cytokine ELISPOT

173. The day prior to the assay coat each well with 50 μl 10 μg/ml purified anti-citokine antibody (e.g., IFN-γ, TNF-α, IL-2, IL-4, IL-10). Incubate overnight at 4° C. Wash plates 4 times with 200 μl PBS. Block wells with 200 μl 10% FCS complete medium, incubate at room temperature for at least 1 hour. Add effector cells to the top wells and make serial dilutions. Discard 100 μl of cells from the last dilution. Add the stimulating antigen and IL-2 to the wells. Incubate plates at 37° C., 5% CO₂ for 16-40 hours (use level to ensure that plates stay even horizontally). Discard cells and wash 5 times with PBS-Tween). Add 100 μl 2 μg/ml biotinylated-anti-cytokine antibody (e.g., IFN-γ, TNF-α, IL-2, ILL4, IL-10) diluted in PBS-Tween. Incubate at room temperature for 2 hours or overnight at 4 C. Wash plate 5 times with PBS-Tween, 200 μl each wash. Add 100 μl 1:1000 Strepavidin-HRP, incubate 1 hour at room temperature. (avoid high background by not exceeding 1 hour incubation). Wash wells 5 times with PBS-Tween. Add 100 μl chromagen substrate per well, incubate at room temperature for 10-20 minutes in dark (at 10 minutes, observe the color developed, if the color is not fully intense, incubate few more minutes and double check). Wash plates under running tap water and air-dry plates (in hood with light turned-off). Count and record numbers of spots/well by ELISPOT plate reader or stereomicroscope (20×).

CTL

174. Direct ex-vivo cytolytic killing is measured in a 5 hr⁵¹Cr release assay. Briefly, target cells are labeled with 51Cr for 1 hr at 37 C. Following the 1 hr incubation, cells are centrifuged for 8 min at 1200 rpm to pellet the cells. The media is poured off and the pellet resuspended in fresh media. Cells are centrifuged and resuspended two additional times. After the third spin, cells are resuspended in an appropriate amount of media to attain the desired effector to target ration (e.g., a 50:1 effector: target ratio with effector cells at a concentration of 1×10⁷/ml would require target cells to be at a concentration of 2×10⁵/ml), and contacted with antigen. Effector cells (cells containing lymphocytes from an immunized animal (e.g., PBL, splenocytes, hepatocytes, and bone marrow) are contacted with the target cells for 5-6 hrs at 37 C. Cells are pelletted by centrifugation and supernatants harvested. ⁵Cr release is measured on an appropriate instrument. Percent killing is (experimental release-spontaneous release/maximum release—spontaneous release)×100.

175. Secondary killing to measure memory cell responses to antigen can also be measured. In this method, effector cells are incubated for 6 days in the presence of antigen stimulation and then used in a cytolytic assay as described above.

### Example 3

176. In accordance with the methods described above the pHSV vector comprising an SIV-Gag-p17/p27 construct was made. This vector is useful to establish a model for the study of the effectiveness of a particular treatment or prophylactic vaccine. For example, such a model can comprise a system to establish a foamy live viral vector for vaccine use. The pHSV with an SIV gag (p17,p27) insert has been tested in vitro and shown to express similar levels of SIV gag as the HIV gag engineered vector. This SIV gag live viral vector can be used to inoculate rhesus macaques in order to determine protective immune responses that can develop following in vivo expression of the vector gene products including the SIV gag protein. Inoculated animals can then be challenged with wild type SIV to further determine any potential vaccine induced efficacy by studying primary (sterilizing immunity) and secondary (time to morbidity) end points.

1. A live replicating human spumavirus vector (pHSV) suitable for human use comprising an immunizing construct, wherein the immunizing construct partially replaces the bet gene:

2. The vector of claim 1, wherein the vector has the sequence of SEQ ID NO:1.

3. The vector of claim 1, further comprising a reporter gene.

4. The vector of claim 3, wherein the reporting gene is GFP.

5. The vector of claim 1, wherein the immunizing construct is an antigen encoding nucleic acid.

6. The vector of claim 5, wherein the antigen-encoding nucleic acid encodes a viral antigen.

7. The vector of claim 6, wherein the viral antigen is an antigen from a virus selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

8. The vector of claim 7, wherein the viral antigen is HIV-GAG p17/p24.

9. The vector of claim 7, wherein the immunizing construct is SIV-GAG.

10. The vector of claim 5, wherein the antigen-encoding nucleic acid encodes a bacterial antigen.

11. The vector of claim 10, wherein the bacterial antigen is an antigen from a bacterium selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

12. The vector of claim 5, wherein the peptide encoding nucleic acid encodes a parasitic antigen.

13. The vector of claim 12, wherein the parasitic antigen is an antigen from a parasite selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

14. The vector of claim 5, wherein the peptide encoding nucleic acid encodes a cancer antigen.

15. The vector of claim 14, wherein the cancer antigen is an antigen from a cancer selected from the group consisting of lymphomas (Hodgkins and non-Hodgkins), B cell lymphoma, T cell lymphoma, leukemias, myeloid leukemia, carcinomas, carcinomas of solid tissues, squamous cell carcinomas of mouth, throat, larynx, lung, head and neck, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, mycosis fungoides, bladder cancer, brain cancer, nervous system cancer, head and neck cancer, kidney cancer, lung cancers such as small cell lung cancer and non-small cell lung cancer, neuroblastoma/glioblastoma, ovarian cancer, liver cancer, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

16. The cancer antigen of claim 15, wherein the antigen is selected from the list of cancer antigen consisting of mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, and melanoma gangliosides.

17. A cell comprising the vector of claim 1.

18. A method of detecting the expression of the vector of claim 1, comprising obtaining a sample containing the vector, contacting the sample with an antibody directed to antigen encoded by the immunizing construct, and detecting antigen bound to the antibody, the presence of bound antigen indicating expression of the vector.

19. The method of claim 18, wherein the sample is blood from a subject.

20. The method of claim 18, wherein the sample is tissue biopsy from a subject.

21. A method of treating a subject with a condition comprising administering to the subject the vector of claim 1.

22. The method of claim 21, wherein the condition is associated with viral infection, and the immunizing construct encodes an antigen from a virus selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type- 1, Hantavirus, Rubella virus, Simian Immunodeficiency virus, Human Immunodeficiency virus type-1, and Human Immunodeficiency virus type-2.

23. The method of claim 21, wherein the condition is associated with bacterial infection, and the immunizing construct encodes an antigen from a bacterium selected from the group sonsisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

24. The method of claim 21, wherein the condition is associated with parasitic infection, and the immunizing construct encodes an antigen from a parasite selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

25. The method of claim 21, wherein the condition is cancer, and the immunizing construct encodes a tumor antigen from the list consisting of human epithelial cell mucin (Muc-1; a 20 amino acid core repeat for Muc-1 glycoprotein, present on breast cancer cells and pancreatic cancer cells), the Ha-ras oncogene product, p53, carcino-embryonic antigen (CEA), the raf oncogene product, gp100/pmel17, GD2, GD3, GM2, TF, sTn, MAGE-1, MAGE-3, BAGE, GAGE, tyrosinase, gp75, Melan-A/Mart-1, gp100, HER2/neu, EBV-LMP 1 & 2, HPV-F4, 6, 7, prostate-specific antigen (PSA), HPV-16, MUM, alpha-fetoprotein (AFP), CO17-1A, GA733, gp72, p53, the ras oncogene product, HPV E7, Wilm's tumor antigen-1, telomerase, and melanoma gangliosides.

26. A method of preventing a condition in a subject comprising administering to the subject the vector of claim 1.

27. The method of claim 26, wherein the condition is associated with viral infection, and the immunizing construct encodes an antigen from a virus selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

28. The method of claim 26, wherein the condition is associated with viral infection, and the immunizing construct encodes an antigen from a virus selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

29. The method of claim 28, wherein the condition is associated with viral infection, and the immunizing construct encodes an antigen from a virus selected from the group consisting of Toxoplasma gondii, Plasmodium falciparum, Plasmodium vivax, Plasmodium malariae, other Plasmodium species., Trypanosoma brucei, Trypanosoma cruzi, Leishmania major, other Leishmania species., Schistosoma mansoni, other Schistosoma species., and Entamoeba histolytica.

30. A method of making a model for use in screening for substances effective in treating or preventing a disease associated with an immunizing construct, comprising administering the vector of claim 1 to an animal capable of manifesting a characteristic of the immunizing construct.

31. A model made in accordance with the method of claim 30.

32. A method of screening for a substance effective in treating a disease associated with an immunizing construct comprising: a) administering the substance to the model of claim 31; and b) assaying for an change in the course of the disease as compared to an the course of the disease in a control subject; an improvement in the course of the disease in the presence of the substance identifies a substance that is effective in treating the disease.

33. A method of screening for an immunizing construct effective in preventing a disease associated with an immunizing construct comprising: a) administering the vector of claim 1 to a subject; b) subjecting the subject to treatment that will induce the disease or characteristic of the disease; and c) assaying for an change in the course of the disease as compared to an the course of the disease in a control subject; an improvement in the course of the disease in the presence of the substance identifies a substance that is effective in preventing the disease.

34. A method of screening for an immunizing construct effective in treating a disease effective in treating a disease associated with an immunizing construct comprising: a) subjecting the subject to treatment that will induce the disease or characteristic of the disease; b) administering the vector of claim 1 to a subject; and c) assaying for an change in the course of the disease as compared to an the course of the disease in a control subject; an improvement in the course of the disease in the presence of the substance identifies a substance that is effective in treating the disease.

35. The method of claim 21, wherein the subject is a cell.

36. The method of claim 21, wherein the subject is a mouse.

37. The method of claim 21, wherein the subject is a monkey.

38. The method of claim 21, wherein the subject is a human.
